SEQUENCE LISTING (9 sequences)

SEQ ID NO: 1 - protein, 16 aa, Artificial
  substrate of Abl kinase, synthesized by addition of CGSN
CGSNEAIYAAPFAKKK

SEQ ID NO: 2 - protein, 15 aa, unknown
  human p53
CSVEPPLSQETFSDK

SEQ ID NO: 3 - protein, 11 aa, unknown
  human c-myc
CEQKLISEEDL

SEQ ID NO: 4 - protein, 23 aa, unknown
  mouse Bad
CTRSRHSSYPNEYEEDEEMEEEL

SEQ ID NO: 5 - protein, 11 aa, unknown
  human c-myc
CEQKLISEEDL

SEQ ID NO: 6 - protein, 15 aa, unknown
  human p53
CSVEPPLSQETFSDK

SEQ ID NO: 7 - protein, 16 aa, artificial
  substrate of Abl kinase, synthesized by addition of CGSN
CGSNEAIYAAPFAKKK

SEQ ID NO: 8 - DNA, 20 bp, unknown
  chemically modified ligand placing a cysteine at the 5-end of DNA
cgctctagaactagtggatc

SEQ ID NO: 9 - protein, 12 aa, artificial
  substrate of Abl kinase
EAIYAAPFAKKK

(19) United States
(12) Patent Application Publication
(10) Pub. No.: US 2004/0198958 A1
(43) Pub. Date: Oct. 7, 2004

(54) Carrier-ligand fusions and uses thereof

(76) Inventors: Ming-qun Xu, Hamilton, MA (US); Inca Ghosh, Somerville, MA (US); Luo Sun, Hamilton, MA (US); Thomas C. Evans, Somerville, MA (US)

Correspondence Address: NEW ENGLAND BIOLABS, INC., 32 TOZER ROAD, BEVERLY, MA, 01915, US

(21) Appl. No.: 10/733,617

(22) Filed: Dec. 11, 2003

Related U.S. Application Data
(60) Provisional application No. 60/432,553, filed on Dec. 11, 2002.
(60) Provisional application No. 60/456,171, filed on Mar. 20, 2003.
(60) Provisional application No. 60/514,934, filed on Oct. 28, 2003.

(51) Int. Cl. C07K 16/18

(52) U.S. Cl. 530/387.1

(57) ABSTRACT

Improved methods for purification, detection or characterization of molecules have been described in embodiments of the present invention. The improved methods rely on the ability to create a carrier reagent which is capable of (i) binding to a matrix either specifically or non-specifically; and (ii) forming a covalent linkage with any ligand having a nucleophilic group or a thioester as a result of a simple reaction which does not require a variety of chemical reagents or sophisticated chemistry. The covalent linkage between the carrier and the ligand relies on the chemical reaction between a thioester and a nucleophilic group. The carrier should either contain a reactive thioester which if it is a protein should be at the C-terminal end of the protein or a reactive nucleophilic group which if it is a protein should be at the N-terminal end and should preferably be a cysteine or selenocysteine. The ligand requires a reactive nucleophilic group to react with the thioester on the carrier or if the carrier has a reactive nucleophilic group, the ligand should have a reactive thioester.

### CROSS REFERENCE

This application claims priority from U.S. application serial No. 60/432,553 filed Dec. 11, 2002; 60/456,171 filed Mar. 20, 2003; and 60/514,934, filed Oct. 28, 2003, herein incorporated by reference.

### BACKGROUND OF THE INVENTION

Methods for purification, characterization and detection of reagents and putative therapeutic molecules such as antibodies, enzymes and receptors are necessary for their use in the biomedical and pharmaceutical industries. These methods utilize attachment of peptide antigens and other molecules to various membranes or affinity columns. However, limitations of these methods occur, for example, because of ineffective binding to affinity substrates. Ineffective binding may result from the small molecular size of the reagent or its weak and inconsistent affinity for membranes or columns which results in low sensitivity, false positives and inconsistency of signals. Furthermore, these methods often employ sophisticated chemistry, which may require the use of various chemical reagents leading to contamination of the environment and increased expense.

### SUMMARY

Improved methods of purifying, characterizing or detecting reagents or putative therapeutic molecules which rely on carrier-ligand molecules are provided herein.

In an embodiment of the invention, a method for purifying a ligand-binding molecule from a mixture is provided that includes: (a) forming a carrier-ligand conjugate by means of a thioester-nucleophile reaction between a carrier reagent and a ligand, the carrier-ligand being optionally immobilized on a matrix; (b) contacting the carrier-ligand conjugate with a mixture containing the ligand-binding molecule; (c) selectively binding the ligand-binding molecule to the ligand; and (d) eluting the ligand-binding molecule from the immobilized ligand so as to obtain the purified ligand-binding molecule.

In a preferred embodiment of the method, the ligand-binding molecule may be any of an antibody, receptor, receptor-binding molecule, enzyme or enzyme substrate. The ligand may be an antigen and the carrier may be capable of binding specifically or non-specifically to a matrix. Where the carrier is specific for a matrix, it is here called a matrix-binding molecule.

A matrix-binding molecule may be selected from a carbohydrate-binding molecule such as a monosaccharide-binding domain, a disaccharide-binding domain, an oligosaccharide-binding domain, a chitin-binding domain, a maltose-binding domain, an arabinose-binding domain, an arabinogalactan-binding domain, a lectin-binding domain or selected from a vitamin-binding domain, a nucleic acid-binding domain, an amino acid-binding domain, a metal binding-domain, a receptor-binding domain, a sulfate binding-domain and a phosphate binding-domain. In a particular preferred embodiment, the matrix-binding protein is a chitin-binding protein.

Where a carrier is capable of non-specific binding to a matrix, the carrier may additionally include M.Hha, paramyosin, chitin-binding domain and maltose binding protein. In these circumstances, the matrix may include nitrocellulose, nylon, SDS-polyacrylamide gel, or a synthetic polymer such as a polystyrene micro-titer plate.

In additional embodiments of the invention, a ligand-binding affinity matrix and method for forming the affinity matrix for binding a ligand-binding molecule is provided. The affinity matrix includes a carrier conjugated to a ligand by means of a thioester-nucleophilic interaction between a nucleophilic N-terminal cysteine or selenocysteine on one of the carrier or ligand and a C-terminal thioester on the other, the carrier-ligand conjugate being immobilized on a matrix, such that the ligand in the carrier-ligand is optionally capable of reversibly binding a ligand-binding molecule.

The method for making the affinity matrix includes: (a) forming a C-terminal thioester by cleavage of a fusion protein wherein the fusion protein comprises a carrier fused to an intein or a ligand fused to intein, such that cleavage occurs in the presence of a thiol reagent at the intein junction with the carrier or the ligand; (b) combining in a mixture, either (i) the carrier with the C-terminal thioester and the ligand with an N-terminal cysteine or selenocysteine or (ii) the carrier with an N-terminal cysteine or selenocysteine and the ligand with the C-terminal thioester; and (c) permitting the carrier to bind the matrix to form, with the ligand after ligation, the affinity matrix.

In an additional embodiment of the invention, a method is provided for screening for the interaction of one or more immobilized ligands with one or more ligand-binding proteins in a preparation. The method includes (a) covalently linking a carrier to a ligand by means of thioester-nucleophilic interaction to form a carrier-ligand conjugate; (b) permitting the carrier-ligand to be immobilized by a matrix; (c) reacting a preparation containing one or more ligand-binding proteins to the carrier-ligand; and (d) detecting the binding of the one or more ligand-binding protein with the one or more immobilized ligands.

In another preferred embodiment, the carrier-ligand may be one type of fusion protein in a set of fusion proteins containing many types of fusion protein where each type is distinguished by a different ligand fused to the same carrier, the fusion proteins being located on the matrix in an ordered array for detecting interactions between ligand and ligand-binding molecules.

In an additional embodiment, the ligand is subject to post-translationally modification such that the ligand-binding domain is specific for the modified ligand. In addition, the ligand may carry a modification such that the ligand could not be translated from a DNA sequence in vitro using the 21 naturally occurring amino acids.

In an additional embodiment, a method is provided for enhancing the immunogenic properties of a ligand such as a peptide antigen in a animal, that includes (a) forming a carrier-ligand fusion protein by intein-mediated ligation; and (b) administering an effective dose of the carrier-ligand fusion protein to the animal to obtain an enhanced immune response compared with the ligand in the absence of the carrier.

### DESCRIPTION OF THE DRAWINGS

The invention will be more readily understood by reference to the following description in which:

FIG. 1 shows the use of intein-mediated protein ligation (IPL) for generating an affinity matrix. A fusion protein precursor (CBD-intein-CBD*), consisting of a chitin-binding domain (CBD) fused to the Mycobacterium xenopi GyrA intein (Mxe) and a second binding deficient CBD mutant where the mutation is W687F mutation (CBD*), was purified from a cell extract by binding the wild-type CBD-portion to a chitin resin. Intein-mediated ligation was achieved by incubating the CBD-intein fusion protein in the presence of 40 mM 2-mercaptoethane-sulfonic acid (MESNA) to induce cleavage of the protein bond prior to the N-terminal cysteine (Cys1) of the intein, resulting in the formation of a reactive thioester on the C-terminus of the wild-type CBD. The CBD with a reactive thioester at the C-terminus was ligated to a synthetic peptide containing an N-terminal cysteine.

FIG. 2 shows confirmation of CBD-peptide formation. The process was monitored by SDS PAGE stained with Coomassie brilliant blue. CBD-intein-CBD* fusion protein was expressed in E. coli and purified by chitin column chromatography.

Lane 1: 1 μl of chitin purified CBD-intein-CBD*

Lane 2: 1 μl of the chitin column after MESNA induced cleavage

Lane 3: 5 μl of the chitin column after cleavage and washing

Lane 4: 5 μl of the ligation product between CBD and hemagglutinin (HA) peptide antigen (CBD-HA)

Lanes 5-7: 5 μl of a chitin column after a wash with 100 mM glycine buffer at various pHs were tested to determine whether CBD-HA remained bound to the column; (lane 5: pH 3.5; lane 6: pH 2.5 and lane 7: pH 1.8)

Lane 8: 5 μl of resin was taken from the column after the washing to confirm that CBD-HA remains bound to the column

FIG. 3 shows a Western Blot analysis establishing that purified anti-HA antibody at a dilution of 5000 fold detects MBP-HA antigen fusion protein (200 ng)

3A: Analysis of rabbit antibodies purified by a CBD-HA column and obtained from the eluant of the column (lane 1) and from flow through serum (lane 2)

3B: Analysis of rabbit antibodies purified by a conventional conjugated HA column and obtained from the eluant of the column (lane 3) and from flow-through serum (lane 4).

FIG. 4 shows an enzyme-linked immunosorbent assay (ELISA) to compare the titer of purified antibody from CBD-peptide columns made using intein-mediated ligation and from conventional conjugated peptide columns. Three rabbit anti-sera (A: anti-HA; B: anti-p53 peptide; C: anti-myc) and one mouse anti-serum (D: anti-Bad peptide) were used in these experiments. Each data point represents the average from three experiments with the standard deviation indicated. In each set of experiments, the relative activity was obtained by arbitrarily designating the highest OD₄₁₄ reading as 1 and converting the rest of the data accordingly.

FIG. 5 shows comparison of the efficiency of a CBD-p53 peptide column to commercially available affinity columns. Activities of antibodies at 1:1000 purified from a CBD-p53 peptide affinity column (panel A, lane 1), Sulfolink-p53 conjugated peptide column (panel B, lane 3) and Aminolink-p53 conjugated peptide column (panel C, lane 5) were examined in Western Blot analysis using an MBP-p53 fusion as a substrate. Antibody activities in the flow through fractions from each column were also examined by a Western Blot assay (1000-fold dilution) to compare their binding efficiencies (lanes 2, 4, and 6).

FIG. 6 shows that the CBD-peptide affinity column is specific for HA antibody or for myc antibody. A 1:1 mixture of anti-HA and anti-myc antibody were passed through CBD-HA (panel A) and CBD-myc columns generated by intein-mediated ligation (panel B). Purified antibodies (lane 1 and 2 in both panels) and flow through factions (lanes 3 and 4 in both panels) were collected and analyzed in Western Blots at a 5000-fold dilution against the following substrates: MBP-myc (lanes 1 and 3 in both panels) and MBP-HA (lanes 2 and 4 in both panels) where myc and HA are the peptide antigens.

FIG. 7 shows the results of ELISA used to determine the antibody titer of a polyclonal anti-p53 antibody and a polyclonal anti-Bad antibody preparation. Both immunogens were generated by IPL where the peptides p53 and Bad were ligated to paramyosin. The sera was diluted twofold to generate a dilution series x-axis). Each data point represents the average from three experiments with the standard deviation indicated. 7A: Polyclonal anti-p53 antibody preparation was obtained by immunizing rabbits with a paramyosin-p53 immunogen. 7B: Polyclonal anti-Bad antibody preparation was obtained by immunizing mice with a paramyosin-Bad immunogen.

FIG. 8 shows a Western Blot analysis of a carrier-antigen fusion created byintein-mediated ligation. The methylase from Haemophilus heaemolyticus (M.HhaI) was obtained from the M.HhaI-intein-chitin-binding domain fusion protein using a thiol reagent, such as MESNA, for cleaving the intein from the M.HhaI protein. The purified M.HhaI with a C-terminal thioester (panel A, lane 1) was then ligated to the peptides p53 and Bad. The ligation products, M.HhaI-p53 (panel A, lane 2) and M.HhaI-Bad (panel A, lane 3) were visualized by Coomassie blue staining of a SDS-PAGE. The same three proteins (M.HhaI, M.HhaI-p53 and M.HhaI-Bad) were used for analysis of the anti-p53 antibody (panel B) used at 1:15,000 and anti-Bad antibody (panel C) used at 1:7,500.

FIG. 9 shows an array of carrier-peptide antigens on a membrane where the carrier-peptide antigens were generated by intein-mediated protein ligation. Peptide antigens with an N-terminal cysteine were ligated to a carrier protein possessing a C-terminal thioester to form the carrier-peptide antigen. The carrier-peptide antigen was diluted 3× serially from the first row to the last row and spotted onto a membrane using a Bio-dot micro-filtration apparatus (Biorad, Hercules, Calif.). The membrane was subjected to an antibody.

Unligated peptide antigen samples were used as controls.

FIG. 10 shows a protein blotting analysis of membrane bound carrier-peptides using peptide-specific polyclonal antibodies.

10A: 0.45 μm nitrocellulose membrane with samples Haemophilus haemolyticus methylase (M.HhaI) (Hha, column 1), paramyosin (column 2), haemaglutinin (HA) peptide (column 3), myc peptide (column 4), 10× myc peptide (column 5), Hha-myc (column 6) and paramyosin-myc (column 7) were incubated with anti-myc antibody (1:5000 dilution). Each sample of column 5 contains 10 fold more myc peptide compared to corresponding samples in column 4. Western Blot analysis was completed using HRP conjugated anti-rabbit antibody and the Lumiglo reagent (Cell Signaling Technology, Inc., Beverly, Mass.).

10B: 0.45 μm nitrocellulose membrane with samples Hha (column 1), paramyosin (column 2), HA peptide (column 3), 10× HA (column 4), myc peptide (column 5), Hha-HA (column 6) and paramyosin-HA (column 7) were Blotted with anti-HA antibody (1:5000 dilution). Each sample of column 4 contains 10 fold more HA peptide compared to the corresponding samples in column 3.

FIG. 11 shows protein blotting assays using different membranes: A. 0.2 μm nitrocellulose; B. 0.2 μm nylon. Carrier-peptide conjugates and unligated peptides were tested against peptide-specific antibodies.

11A: Membrane was dotted with the samples paramyosin (column 1), HA peptide (column 2), myc peptide (column 3), p53 peptide (column 4), and paramyosin ligated to the peptides, HA (column 5), myc (column 6) and p53 (column 7).

11B: Membrane was dotted with samples, M.HhaI (Hha, column 1), HA (column 2), myc (column 3), p53 (column 4), and M.HhaI (Hha) ligated to the peptides, HA (column 5), myc (column 6) and p53 (column 7). Both membranes were incubated with a mixture of antibodies of anti-HA (1:5000), anti-myc (1:5000), and anti-p53 (1:5000), before being reacted with secondary antibody and visualized using the Lumiglo reagent (Cell Signaling Technology, Inc., Beverly, Mass.).

FIG. 12 shows a screening assay for carrier proteins used in carrier-peptide conjugates for enhancing the sensitivity of antibody recognition of peptides. Carrier proteins M.HhaI (Hha), MBP, paramyosin and CBD were ligated with HA peptide. Protein blotting was performed using a 0.45 μm nitrocellulose membrane with HhaI (column 1), MBP (column 2), paramyosin (column 3), CBD (column 4), HA peptide (column 5), Hha-HA (column 6), MBP-HA (column 7), paramyosin-HA (column 8), CBD-HA (column 9) and subjected to polyclonal rabbit anti-HA antibody detection (1:5000).

FIG. 13 shows retention of a fluorescent peptide (FluP) on a membrane. FluP, consisting of amino acids CDPEK*DS (* is the fluorescent label) (New England Biolabs, Inc., Beverly, Mass.), was spotted on a nitrocellulose membrane (0.2 μm). FluP was ligated to the thioester-tagged proteins, M. HhaI (Hha; column 1), MBP (column 2), Paramyosin (column 3), and CBD (column 4). The unligated FluP peptide was used as a control (column 5).

13A: Before mock Western Blotting

13B: After mock Western Blotting

FIG. 14 shows the result of alanine scanning of a HA epitope using a carrier-HA conjugate where the carrier is M.HhaI (Hha). The conjugate was diluted threefold from row 1 to row 4 (conjugated HA) and from row 5 to row 9 (unconjugated HA) was also diluted threefold. Peptides P1-P8 with alanine mutations and P9 (Panel A) were ligated to M.HhaI (Hha). The resulting Hha-HA (rows 1-4) were spotted onto nitrocellulose membrane (0.45 μm) along with the unligated HA peptide (rows 5-8) (Panel B). The membrane was subjected to Western Blotting using monoclonal anti-HA antibody (1:5000 dilution, Cell Signaling Technology, Inc., Beverly, Mass.).

FIG. 15 shows the results of an ELISA performed using:

15A: Peptide HA and Paramyosin-HA

15B: Peptide HA and M.HhaI-HA (Hha-HA)

15C: Peptide myc and Paramyosin-myc

15D: Peptide myc and M.HhaI-myc (Hha-myc)

Each data point represents the average, normalized OD value of 9 readings acquired from three independent experiments. The relative activity was obtained by arbitrarily designating the highest OD₄₁₄ value from each reading as “1” and normalizing the data accordingly. The error bars for each point represent the standard deviation for the data, which incorporates experimental uncertainty as well as the measurement uncertainty of the spectrophotometer. The x-axis is a logarithmic scale, while the y-axis is a linear scale. Antibodies in the first row were used at a dilution of 1/1000 and subsequently diluted twofold for the next row. Each row was diluted twofold to generate a dilution series.

FIG. 16 shows a flow chart of the production of substrates for analysis of protein modification. The flow chart describes how a protein with putative phosphorylation sites can be analyzed using IPL. Peptides derived from the amino acid sequence of the protein are synthesized with a N-terminal cysteine and then ligated to a carrier protein (1). The ligated product can be treated with a kinase or kinases (2). The phosphorylated protein can be detected by Western Blot analysis of the ligated protein using a phospho-specific antibody (3).

FIG. 17 shows how a phosphorylated protein can be detected by Western Blot analysis of the ligated protein using a phospho-specific antibody (anti phospho-tyrosine antibody 1:2000; Cell Signaling Technology, Inc., Beverly, Mass.). The peptide CGSNEAIYAAPFAKKK (1697 Da) (SEQ ID NO:1) (Songyang, Z. et al., Nature 373:536-539 (1995)) treated with Abl Protein Tyrosine Kinase (New England Biolabs, Inc., Beverly, Mass.) was ligated to a carrier selected from paramyosin (29 kDa); M.HhaI (39 kDa); and MBP (43 kDa). The carrier-ligand was subjected to Western Blot analysis using the anti-phospho-tyrosine antibody. Only the lanes where the ligated product was treated with Abl kinase gave a detectable positive signal (lanes 2, 3, 8, 9, 14 and 15). The controls of the carrier alone (lanes 5, 11 and 12), ligated protein with no kinase (lanes 1, 7, 16) and kinase alone (lane 6) gave no signal indicating that the phosphorylation was specific for the peptide.

FIG. 18 shows an example of how carrier-antigen conjugates formed by intein-mediated ligation are used for antibody purification and analysis. Generation of a CBD possessing a C-terminal thioester by the IMPACT™ (New England Biolabs, Inc., Beverly, Mass.) system permits its ligation with a peptide antigen to create a CBD-peptide fusion for binding to a chitin column, forming an affinity column for purifying antibody raised against the same peptide antigen. The antibody raised by immunization of rabbit (or other animal) after its conjugation to keyhole limpet hemacyanin (KLH) or another carrier protein can be purified by passage of the anti-sera through this affinity column. This single column purification method has its advantage over the use of protein A column and peptide column generated by chemically conjugated method. The purified antibody can be analyzed using the same antigen ligated to a different carrier protein produced by IMPACT™ (New England Biolabs, Inc., Beverly, Mass.) system.

FIG. 19 shows a general scheme of a carrier-ligand in the context with the matrix and the ligand-binding molecule: (1) refers to matrix; (2) refers to a carrier; (3) refers to a ligand; (4) refers to a ligand-binding molecule; and (5) refers to a peptide-bond linkage between a carrier and a ligand.

Improved methods for purification or characterization of molecules have been described in embodiments of the present invention. The improved methods rely on the ability to create a carrier reagent which is capable of (i) binding to a matrix either specifically or non-specifically; and (ii) forming a covalent linkage with any ligand having a nucleophilic group or a thioester as a result of a simple reaction which does not require a variety of chemical reagents or sophisticated chemistry. The covalent linkage between the carrier and the ligand relies on the chemical reaction between a reactive thioester and a reactive nucleophilic group. If the carrier is a protein, then either the thioester should be at the C-terminal end or the nucleophilic group should be at the N-terminal end of the protein. Accordingly, the ligand should contain a nucleophilic group to react with the thioester on the carrier; if the carrier has a reactive nucleophilic group, the ligand should have a reactive thioester.

The use of a reagent carrier capable of forming a conjugate to any ligand by means a nucleophilic-thioester linkage provides new opportunities for rapid and efficient high throughput screening of libraries of ligands or ligand-binding molecules. Other uses include: various improved immunological assays such as a single step purification for antibodies; improved immunogens for raising an antibody response; and improved ELISA and Dot Blot assays where the improvement includes at least one of: an enhanced positive signal; or reduced background signal. Additional uses of carrier-ligand conjugates as described above further include rapid screening for a capability for post-translational modifications.

In preferred embodiments of the invention, the methods rely on the use of carrier-ligands that are demonstrated to have at least one of the following advantages: (i) size, in visualizing a reaction with an antibody on a substrate such as in a Western Blot; (ii) absence of a requirement for purification of multiple proteins for ligand array screens; (iii) consistency of binding efficiency of a carrier-ligand to a matrix; (iv) the increased retention of a ligand in a carrier-ligand conjugate on the matrix; and (v) the ability to quantify the concentration of a carrier-peptide (Examples I-VI).

Embodiments of the method have advantages over the prior art. In the prior art, a covalent linkage (conjugates) may be formed between carriers and ligands where these are peptides or proteins or a mixture of the two using recombinant techniques to ligate DNA, expressing the fusion molecule and transforming host cells to express the fusion protein. Examples of this type of fusion molecule and methods of making the same are provided in U.S. Pat. No. 5,643,758, International Publication No. WO 03/087301 and in the New England Biolabs, Inc., Beverly, Mass. catalog). This approach requires the cloning and expression of each fusion protein of interest which may under certain circumstances be laborious.

A second approach involves chemical ligation. For example, a ligand such as a peptide antigen is conjugated via its carboxy- or amino-terminal residue to agarose beads or through chemical conjugation of free amino, sulfhydryl, phenolic or carboxylic acid groups (Briand, et al., J. Immunol. Methods 78(1):59-69 (1985); Hinman, et al., Mol. Immunol. 22(6):681-688 (1985); Domen, et al., J. Chromatogr. 510:293-302 (1990); Lundbald, Chemical Reagents for Protein Modification, 2nd ed. CRC Press, Boca Raton, Fla. (1991); Wong, Chemistry of Protein Conjugation and Crosslinking, CRC Press, pp. 248-251 (1991); Hermanson, et al., Immobilized Affinity Ligand Techniques, pp. 75, 99, 118, 143 (1992)).

The method of linking different proteins by intein-mediated ligation was described in International Publication Nos. WO 01/57183, WO 00/47751, WO 00/18881 and WO 00/71701; Evans et al. Biopolymers 51(5):333-42 (1999); Evans et al. J. Biol. Chem., 274:3923-3926 (1999); Evans et al. J. Biol. Chem. 274:18359-18363 (1999); and Muir et al. P.N.A.S. 95:6705-6710 (1998) following from the earlier work described in U.S. Pat. Nos. 5,496,714 and 5,839,247. Purification of proteins by intein cleavage of a tripartite carrier-intein-antigen molecule was described in U.S. Pat. No. 5,834,247.

The use of the following terms as provided below refer to particular embodiments of the invention.

The term “protein” includes a molecule formed from a plurality of amino acids including polypeptides and oligopeptides, and further includes natural or non-natural modifications or derivatives thereof including short synthetic peptides.

The term “carrier” refers to a molecule that is capable of binding specifically or non-specifically to a matrix. A carrier that is capable of binding specifically to a matrix may here be referred to as a matrix-binding molecule.

In a preferred embodiment, the matrix-binding molecule remains bound to the matrix under conditions where a ligand-binding molecule can be eluted from a ligand which is covalently attached to the matrix-binding molecule.

Examples of matrix-binding molecules include: a monosaccharide-binding domain, a dipolysaccharide-binding domain, an oligosaccharide-binding domains, a chitin-binding domain, a maltose-binding domain, an arabinose-binding domain, a cellulose-binding domain, an arabinogalactan-binding domain, a lectin-binding domain, a vitamin-binding domain such as avidin, nucleic acid-binding domains, amino acid-binding domains, metal binding-domains, receptor binding-domains, sulfate binding-domains and phosphate binding-domains. Preferably, the matrix-binding domain may be a sugar-binding molecule such as a chitin-binding domain, a maltose-binding domain and a cellulose-binding domain from a variety of sources.

The term “matrix” refers to any three dimensional structure suitable for immobilizing a carrier. The carrier may be immobilized on the surface or within the matrix. The matrix may include for example, beads, columns, papers, glass, gel or other solid substrate. The matrix may be wholly or partially composed of a naturally occurring polymer such as a carbohydrate, a protein, a lipid or a synthetic polymer. Examples of a matrix material include a sugar such as chitin, maltose or cellulose (U.S. Pat. Nos. 5,643,758 and 5,496,247).

The term “ligand” includes any molecule which is capable of being recognized by a second molecule which has a binding affinity for the ligand. The second molecule may be an antibody but may also be a receptor, antagonist or agonist and may be a biological macromolecule or a synthetic or naturally occurring small molecule such as a synthetic peptide. A ligand may be a protein, a DNA, a lipid, a carbohydrate or a small molecule.

The term “antibody” includes monoclonal or polyclonal antibodies and also single chain antibodies, chimeric antibodies and antibody fragments.

A “nucleophile” preferably contains a free amino group and a free sulfhydryl group. Examples of nucleophilic groups are cysteine and selenocysteine. When the nucleophilic group is an amino acid, it may be attached to a non-protein molecule by a bridge molecule such as, for example, cys-aminohexyl-. The nucleophilic group can be added to a ligand or carrier by any of (a) recombinant means, (b) synthetic means or (c) by intein cleavage. Where recombinant means are utilized, a fusion protein can be expressed in a host cell which is then cleaved in the presence of a protease. For example, a carrier-Factor X fusion can be cleaved by Factor X protease to yield a carrier having an N-terminal cysteine. If Factor X is additionally fused to a matrix-binding protein, the fusion protein can be first purified by affinity column purification prior to cleavage. In place of Factor X, an enzyme such as genenase may be similarly used for cleavage of a carrier-genenase-matrix-binding protein fusion. The process of intein cleavage to generate an N-terminal cysteine or selenocysteine is further described in International Publication No. WO 00/47751. Synthesis of molecules with a nucleophilic group may be achieved by any known type of chemical synthesis including the method of Example VIII.

The term “intein” refers to a self-splicing protein and includes any of the inteins provided in InBase™, from New England Biolabs, Inc. (Beverly, Mass.) at (InBase, New England Biolabs Intein Database:www.neb.com/neb/inteins.html.) An intein further includes derivatives or modifications of the listed inteins including amino acid substitutions as long as these derivatives and modifications permit cleavage. The DNA encoding these inteins have been described and it is known that certain mutations of these intein sequences do not prevent an intein induced cleavage reaction.

The term “thioester” refers to a structure in which a carboxylic acid and a thiol group are linked by an ester linkage or where a carbonyl carbon forms a covalent bond with a sulfur atom (see below).

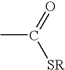

A thioester can be formed by replacing the nitrogen atom of an amide bond by a sulfur atom in an intein-catalyzed reaction. A thioester structure (—COSR1) can be substituted by another thioester (—COSR2) as shown in an intein-mediated ligation reaction. A reactive thioester may be added to a protein or non-protein molecule by chemical synthesis.

The term “thiol reagent” refers to a small molecule containing a sulfhydryl group. Thiol reagents may be identified by searching a commercial reagent catalog of the type provided by Sigma, St. Louis, Mo. In addition, particular thiol reagents have been identified as effective for intein cleavage. These include: thiophenol and dithiothreitol. A preferred thiol reagent is MESNA which was found unexpectedly and is described in detail in International Publication No. WO 00/47751.

The term “ligand-binding molecule” refers to any molecule that binds to a ligand by non-covalent means. Ligand-binding molecules include proteins, DNA, RNA, lipids, carbohydrates and may be biological macromolecules, small biological molecules or synthetic small molecules. In particular instances, the ligand-binding molecule may be an antibody, receptor protein, a second antigen or any type of protein known to bind a ligand.

Use of Carrier-Ligands in a One Step Purification of Ligand-Binding Molecules

Ligand-binding molecules are widely used in biomedical research and pharmaceutical applications, such as in identification and cloning of new genes, purification and structure-function analyses of proteins, identification of ligands such as antigens, immunohistochemical localization, classification and identification of cell types, as well as disease diagnosis and treatment (Yelton and Scharff, Annu. Rev. Biochem. 50:657-680 (1981); Abell and Denney, J. Natl. Prod. 48(20):193-202 (1985); Eisenbarth, Anal. Biochem. 111(1):1-16 (1981)). In the example of antibodies, purified antibodies give clearer results than crude animal anti-sera in Western Blot analyses, ELISA and immunohistochemical staining and they are essential for the elimination of false positives in medical diagnosis and the avoidance of adverse effects in medical treatments (Gonyea, Clin. Chem. 23 (2 Pt. 1):234-236 (1977); Jiskoot, Mol. Immunol. 124(1):143-156 (1989)).

For the purposes of this section, the carrier is identified as a matrix-binding molecule because of its specific binding to the matrix.

To purify a ligand-binding molecule in a mixture, the ligand-binding molecule is selectively immobilized by binding to a ligand which is itself conjugated to a matrix-binding molecule which in turn is attached to a matrix. In a preferred embodiment, the matrix-binding molecule is bound to a matrix column or to beads.

Immobilization of a ligand-binding molecule occurs in a single simple and efficient step as does the elution of the ligand-binding molecule from the immobilized ligand. Elution of the immobilized ligand-binding molecule does not rely on intein cleavage but rather on changing buffer conditions to disrupt the association of the ligand-binding molecule with the ligand.

Any matrix-binding domain exhibiting an affinity to a matrix, such as chitin-binding domain for chitin beads, that is not disrupted under conditions for elution of a ligand-binding molecule, such as an antibody, can be selected for formation of the matrix for purifying the ligand-binding molecule(s). Conditions of elution of the ligand-binding molecule may include use of a buffer having a pH in a range from pH 1.5 to pH 7. For example, CBD-peptide binds to a chitin matrix under different pH conditions so that, for example, elution of a ligand-binding domain exhibiting a high affinity to a ligand can occur in a buffer with a pH as low as 1.8, or at pH 2.5-3 without disrupting the CBD binding to the chitin matrix. Example I and FIG. 18 show how a CBD-peptide column is used as a matrix for purification of antibodies with affinity to a peptide antigen.

A ligand-binding molecule, for example, an antibody, may be purified from a mixture, for example, a serum, using binding, washing and elution steps. In general, a standard protocol is followed to allow the ligand-binding molecule to first interact with its ligand for example an antigen while the other substances present in the mixture are washed off the affinity matrix. Then the ligand-binding molecule is eluted under conditions such as pH 1.8-3.0 in which the ligand-binding molecule is released from the affinity matrix while preserving the binding of the affinity binding domain-ligand fusion to the resin.

A CBD-ligand affinity column formed by intein-mediated ligation has a comparable binding efficiency for ligand-binding molecules to that reported for affinity matrices generated by chemical conjugation methods as exemplified for purifying antibodies from either rabbit or mice antisera.

The purification method described above can be used for the purification of any ligand-binding molecule. Particular examples of ligand-binding molecules include an antibody or an antigen; a receptor-binding molecule or a receptor; or an enzyme or enzyme substrate.

High Through-Put Screening of Ligand-Binding Molecules or Ligands

High through-put screening may involve testing a large number of ligands or ligand-binding molecules or indeed agonists or antagonists that interfere with binding of ligand to ligand-binding molecules. Examples of agonists or antagonists are small molecules that have a size generally less than 10,000 KDa amino acids and may act as inhibitors or activators to regulate the function of ligand-binding molecules. High through-put screening can be used for a variety of biological assays, as exemplified below for antibody testing, epitope scanning, peptide receptor binding or protein modification assays.

Screening large numbers of samples of ligand or ligand-binding molecules may be achieved by using dot blot analysis, or ELISA. Arrays may be used for antibody epitope and mimitope mapping, mapping of various interactions such as protein-protein, protein-carbohydrate, protein-nucleic acid or protein-lipid interactions and the investigation of enzyme-substrate and enzyme-inhibitor interactions (for example, kinases, proteases, isomerases, chaperones, phosphatase, glycosylases, methylases, acetylases etc.) as well as many specific applications (Reimer, U., et al. Curr. Opin. Biotechnol. 13:315-320 (2002); Reineke, U., et al. Curr. Opin. Biotechnol. 12:59-64 (2001); Templin, M. F., et al. Trends Biotechnol. 20:160-166 (2002); Welfle, K., et al. J. Mol. Recognit. 14:89-98 (2001).

Analysis of arrays depends on binding a ligand such as a protein, peptide or another substrate to a matrix such as a membrane. Problems arise from poor affinity of the ligand for a membrane or other types of matrices as a result of physical and chemical properties such as molecular mass, charge, hydrophilicity, etc. This has hampered the efforts to utilize many types of the commercially available membranes. Poor binding between the ligand and the matrix and differences in binding affinities of different ligands to a matrix result in low sensitivity and false positive results. While not wishing to be bound by theory, the reason for the observed improved properties using carrier-ligands formed by intein-mediated ligation is believed to be the result of improved binding affinity, stronger interaction between the ligand and ligand-binding molecule as well as enhanced retention of the carrier-ligand by the matrix.

Embodiments of the invention include a simple and efficient method for immobilizing ligands such as peptides or other small molecules of interest to a matrix by coupling of the ligand to a carrier. This results in improved sensitivity in the detection of the interaction between the immobilized ligand and ligand-binding molecule. Examples III-VI show how a carrier-ligand can be effectively used for enhanced binding to membranes (Example IV, V), to gels in Western Blots (Example III) and to plastics in ELISA (Example VI).

The specificity of a ligand-binding molecule such as an antibody for a ligand such as an antigen, for example, a synthetic peptide, can be readily determined using Western Blot analysis. A Western Blot involves the transfer of the proteins, in this case the carrier-ligand to a nitrocellulose membrane. The ligand-binding molecule will specifically bind to the ligand immobilized on nitrocellulose and this is visualized using staining, chemiluminescence, fluorescence, radioactivity or other standard detection method. The specific binding of a ligand-binding molecule to the ligand produces a distinct band (FIG. 8). If the ligand-binding molecule does not recognize and bind to the ligand, no band will be visualized on Western Blot analysis. The sharp band is due to a target ligand transferred to the membrane.

The use of Western Blot analysis relies on the ligand being of a certain size which is sufficiently large not to run off the gel during electrophoresis and sufficiently small to enter the gel. Hence it is desirable that the ligand should have a size of at least about 5000 Da.

Whereas it is desirable to couple a large protein to an antigen to enhance an antigenic response, problems with Western Blots occur when the protein carrier is beyond a certain size or is difficult to visualize on a protein gel such in the case of KLH, a widely used carrier molecule for generation of immunogens (450-13,000 kD; Princeton Biomolecules Corporation, Langhorne, Pa.). Using intein-mediated ligation, any carrier in the desired size range having a C-terminal thiol ester or an N-terminal cysteine or selenocysteine can be fused to a peptide antigen with an N-terminal cysteine or selenocysteine or C-terminal thioester, respectively. The fusion protein can be easily visualized by SDS-PAGE, allowing for easy evaluation of the amount of antigen.

When a ligand has a molecular weight size less than the desired size, the ligand is here linked to a carrier which preferably does not interact with the ligand-binding molecule. The carrier molecule should have a molecular weight of a size such that a fusion between the carrier and the selected ligand results in a molecule which will migrate to a convenient predetermined range of positions during SDS-PAGE.

In this section, carrier molecules are preferably proteins, more particularly, moderate to large proteins. Examples of carriers are M.HhaI, paramyosin, MBP, CBD. An optimal carrier protein can be screened for its affinity or binding to a matrix with or without ligation to the small peptide of interest. Ligands described in the examples are peptide antigens (Example IV or V) or peptides for enzyme modification (Example VII).

Ligand arrays such as peptide arrays with increased sensitivity can be produced by immobilization of a peptide using a thioester-tagged carrier molecule. The carrier-peptide conjugate is arrayed or spotted directly onto a matrix such as nitrocellulose, nylon, glass etc. and screened with antibody such that the conjugates that bind the antibody can be detected using a standard detection assay. The improved binding efficiency by the carrier-ligand fusion molecule results in enhanced signal/noise ratio in analysis of ligand-binding molecules that recognize the peptide.

Use of Carrier-Ligands as Immunogens

In those circumstances where the size of the antigen impacts the immune response by a mammal (Harlow and Lane, Antibodies, Cold Spring Harbor Laboratory, Cold Spring Harbor, N.Y. (1988)), forming a carrier-antigen conjugate to enhance the size of the antigen prior to injection of the immunogen into an animal has proved effective as shown in Example II. In order to avoid false positives resulting from cross reactivity, carriers that may be utilized in Western Blots or ELISA as reagents for forming carrier-antigen conjugates are chosen to be immunologically distinct from those used for raising the corresponding antibodies in mammals.

Examples of carrier-antigens that may be used for raising an antibody response in a mammal or alternatively for Western Blot or ELISA analysis of the antisera or purified antibody include MBP, paramyosin and M.HhaI. For example, in Example II, when paramyosin (Δ Sal fragment; Limberger, et al. Mol. Biochem. Parasitol. 38:271-280 (1990)) was used as a carrier protein, an effective immune response against p53 (1.7 Kd) and Bad (2.9 Kd) peptides was obtained (FIG. 7) demonstrating the utility of paramyosin as a carrier protein in the production of an immunogen.

Post-Translational Modification and Analysis of Sites of Modification on a Protein

The activity of proteins may be modulated by post-translational modifications including, for example, phosphorylation, acetylation, methylation, dephosphorylation, glycosylation, deglycosylation, prenylation, vitamin and selenocysteine modifications which occur at specific sites on the proteins. Finding modification sites on a protein offers the opportunity to intervene in protein function.

In an embodiment of the invention, the location of post-translational modification sites has been determined by synthesizing peptides with a sequence that matches the putative modification site, attaching the peptide to a carrier by means of an N-terminal cysteine or selenocysteine: C-terminal thioester bond, reacting the peptide with an enzyme for example a kinase and then analyzing the carrier-modified peptide using antibodies that specifically recognize the modified amino acid and determining a positive reaction on a Western Blot or by other forms of analysis (FIG. 16).

If a peptide does contain a modification site then a positive signal will be detected with a specific antibody in Western Blot analysis or ELISA. In FIG. 17, the unmodified protein-peptide substrate, carrier protein alone or the enzyme (or other reagents) were included as negative controls to show that the modification site was present on the peptide and not on the carrier protein or other reagents present in the assays. Protein modification can be verified by mass spectrometry techniques or other chemical assays.

Formation of a Carrier-Ligand

To generate a fusion of a carrier with a ligand by using intein-mediated ligation, the reactive terminal group on the ligand and the carrier protein may be selected so that (a) if there is a reactive C-terminal thioester on the carrier, then the ligand has a reactive nucleophilic group; or (b) if the reactive terminal group on the carrier is a reactive nucleophile, then the ligand will have a reactive C-terminal thioester group. Where the nucleophilic group is present on a protein, it is preferably a cysteine or selenocysteine at the N-terminal end. In a preferred embodiment, reactive C-terminal thioester on one of the carrier or ligand is the product of intein cleavage in the presence of a thiol reagent. In a preferred embodiment, the reactive nucleophilic group is an N-terminal cysteine or selenocysteine which may be introduced into the carrier or ligand by chemical synthesis or by genetic engineering.

In one embodiment of the invention, a carrier or ligand having a C-terminal thioester may be formed by first cloning a carrier-intein fusion protein or ligand-intein fusion protein where the carrier protein or ligand protein is positioned upstream of the intein (for example, a vector selected from IMPACT™ (New England Biolabs, Inc., Beverly, Mass.) system or designed according to an IMPACT™ (New England Biolabs, Inc., Beverly, Mass.) vector design.

After expression in vivo, and purification, the resulting fusion protein may be cleaved at the junction with the intein to form the carrier or ligand having a C-terminal thioester in the presence of a thiol reagent, such as MESNA (International Publication No. WO 00/47751).

In the examples, a reactive C-terminal thioester of the CBD is generated where the CBD is a matrix-binding domain and where the matrix is chitin. This is described in detail in Example 1. The C-terminal thioester is formed by cleavage of the peptide bond between the CBD from B. circulans and an engineered intein from Mycobacterium xenopi GyrA expressed and purified from E. coli in the presence of the thiol reagent MESNA (U.S. Pat. Nos. 5,496,714 and 5,834,247; U.S. application Ser. No. 09/249,543; Muir, et al., Proc. Natl. Acad. Sci. USA 95(12):6705-6710 (1998); Evans, et al., Protein Sci. 7(11):2256-2264 (1998); Evans, et al., J. Biol. Chem. 274(7):3923-3926 (1999); Evans et al., Biopolymers 51(5):333-342 (1999)).

Where the ligand or carrier has an N-terminal nucleophile, this nucleophile may be a cysteine and selenocysteine. The desired terminal amino acid may be introduced by standard genetic engineering techniques or by synthetic chemistry. For example, a peptide may be synthesized having a terminal cysteine or selenocysteine group using standard peptide synthesis chemistry. An N-terminal cysteine can be created by the IMPACT™-TWIN system (New England Biolabs, Inc., Beverly, Mass.) or by pMal-C2 vector (New England Biolabs, Inc., Beverly, Mass.). Additionally, a cysteine or selenocysteine can be introduced into carrier or ligand by intein-mediated ligation as described above for forming a C-terminal thioester. Instead, intein cleavage occurs at the C-terminal end of the intein in place of the N-terminal cleavage site utilized above.

Conjugation (or ligation) between carrier and ligand occurs when the ligand and the carrier are mixed together in the presence of a thiol reagent.

For purposes of purifying ligand-binding molecules, a matrix-binding domain-intein fusion is first expressed (Example I uses a CBD-intein fusion). The intein is released from the column in the presence of MESNA or other thiol reagent to form the C-terminal thioester on the matrix-binding molecule before or after the absorption of the fusion protein to the matrix. The matrix-binding protein can be ligated to the ligand via a nucleophilic group on the ligand to create a matrix:matrix-binding-domain-ligand column (affinity matrix) for purifying the ligand-binding molecule.

The method of forming the affinity matrix takes advantage of the high-binding affinity of a matrix-binding molecule, such as chitin-binding domain, to a particular molecule such as chitin and the ease of ligation of the matrix-binding domain to the ligand to create an affinity matrix for reacting with a ligand-binding molecule.

For creating a carrier-ligand array, dot Blots, or Western Blots, a carrier-intein-CBD fusion was expressed in a host cell transformed with a vector encoding the protein. The protein product was absorbed onto a chitin column. The carrier which was not limited to CBD, containing a C-terminal thioester was released from the chitin column in the presence of MESNA or other thiol reagents. This carrier could then be ligated to any ligand (peptide antigen) possessing a N-terminal cysteine to generate a covalently linked carrier-ligand fusion.

The individual components of the carrier-ligand may be expressed in any suitable eukaryotic or prokaryotic cells such as yeast, insect cells and mammalian cells. Alternatively, if the ligand is not a protein, the terminal nucleophilic group or thioester can be added to the ligand molecule using synthetic techniques known in the art.

The described embodiments of the invention are intended to be merely exemplary and numerous variations and modifications will be apparent to those skilled in the art. All such variations and modifications are intended to be within the scope of the present embodiments as defined in the appended claims.

The references cited above and below are herein incorporated by reference.

### EXAMPLE I

### CBD as a Matrix-Binding Molecule for Forming an Antibody Affinity Matrix

This example demonstrates the use of a matrix-binding molecule, the chitin-binding domain for immobilizing an antigen ligand to chitin for affinity purification of antibodies specific for the antigen.

Construction and Expression of a Chitin-Binding Domain-Intein Fusion Gene

The first step of the process involves cloning of the CBD to produce a CBD-intein fusion protein using an IMPACT™ (New England Biolabs, Inc., Beverly, Mass.) vector.

CBD (also referred to as B) is derived from Bacillus circulans WL-12 chitinase A1 gene and was cloned in frame to the N terminus of the Mycobacterium xenopi GyrA intein (X) followed by a binding deficient mutant CBD (B*) carrying a W687F mutation (CBD*;B*). pXBX* was constructed by cloning a 0.8 kb XhoI-PstI fragment from pPXB (W687F) (Ferrandon et al. BBA 1621:31-40 (2003)) into the pBSC vector (Mathys, et al., Gene 231(12):1-13 (1999). pBXB* expresses a tripartite fusion protein (BXB*). The W687F mutation in the C-terminal CBD abolished its chitin-binding activity. Protocols for cloning may be found in Sambrook, et al., Molecular Cloning, sections 1.53-1.72 (1989); and Ausubel, et al., Current Protocols in Molecular Biology (1996).

Production and Purification of the CBD Having a C-Terminal Thioester

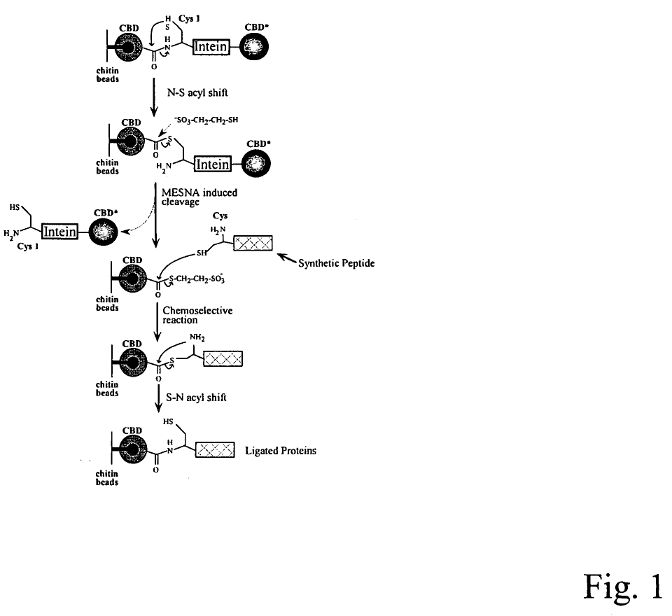

The following experiment demonstrates a procedure for producing an affinity binding domain possessing a C-terminal thioester (FIG. 1: (1) and (2)).

Escherichia coli strain ER2566 (New England Biolabs, Beverly, Mass.) was transformed with the pBXB plasmid and grown at 37° C. in 1 liter of LB medium containing 100 μg/ml ampicillin. Expression of the CBD-X-CBD* fusion protein was induced overnight at 15° C. by adding 0.3 mM isopropyl-β-D-thiogalactoside (IPTG) after the cell density had reached an OD₆₀₀ of 0.5. Induced cells were pelleted by centrifugation and resuspended in 0.5 M NaCl, 20 mM Tris-HCl, pH 8.5. Following sonication, cell debris was removed by centrifugation at 4000×g for 30 minutes. Clarified supernatants were loaded at 4° C. onto a column containing 20 ml insoluble chitin beads (New England Biolabs, Beverly, Mass.) followed by a wash of 10 column volumes of column buffer (0.5 M NaCl, 20 mM Tris-HCl, pH 8.5), resulting in purified CBD-X-CBD* bound on to the chitin beads (Current Protocols in Protein Science, Eds. Coligan, J. E., et al., Pub. John Wiley and Sons, 1997)

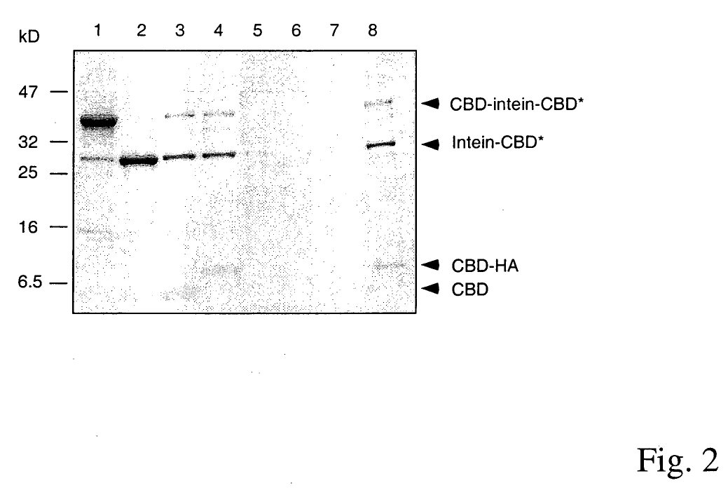

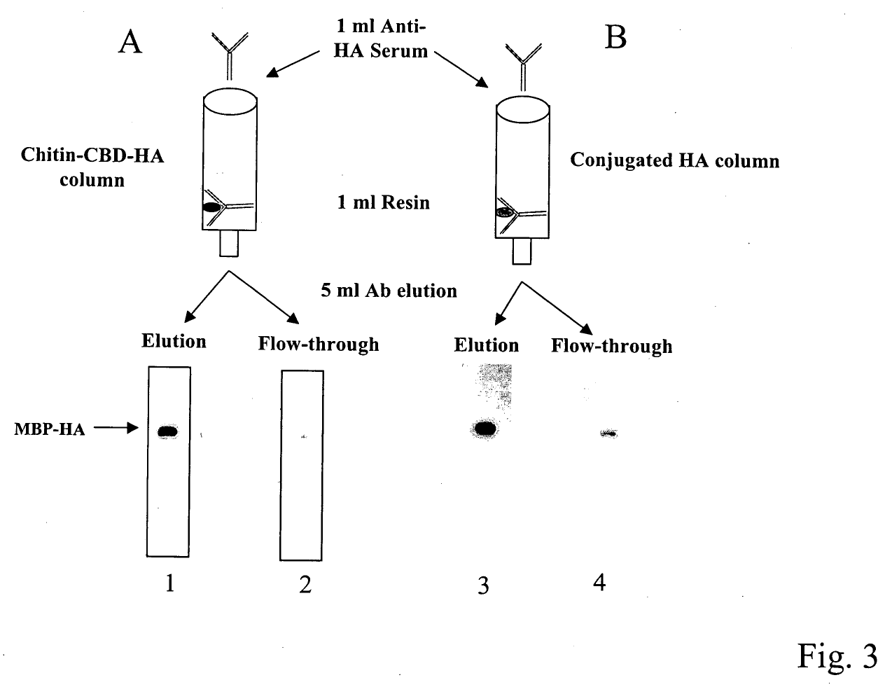

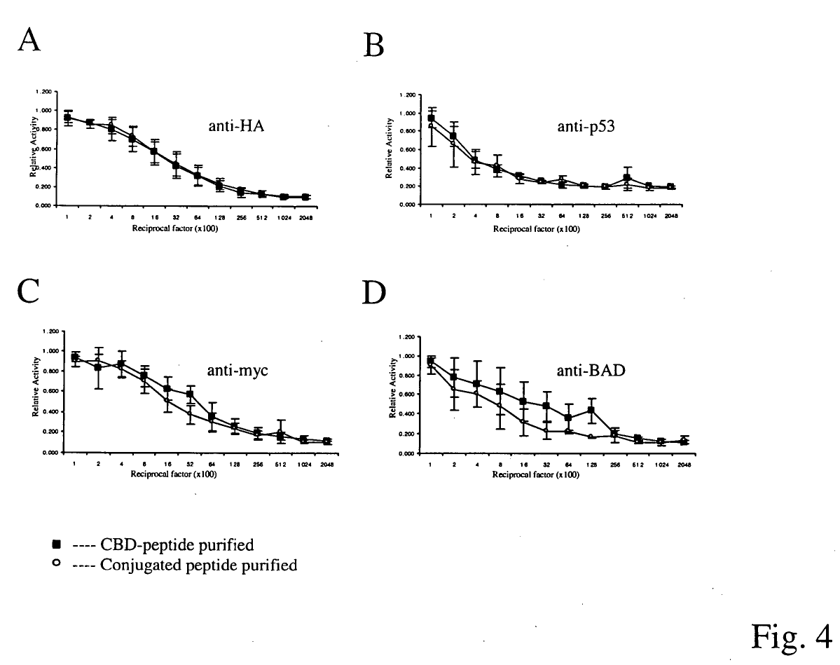

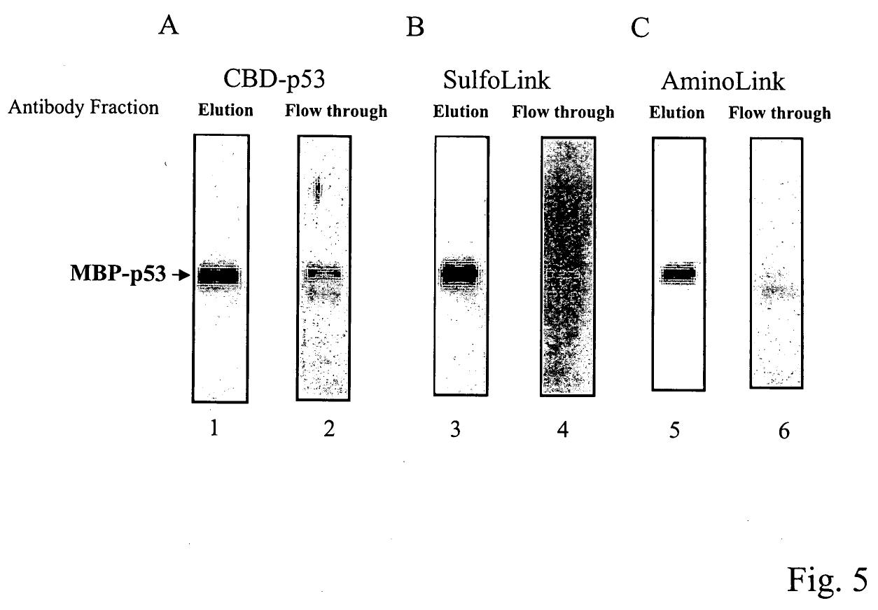

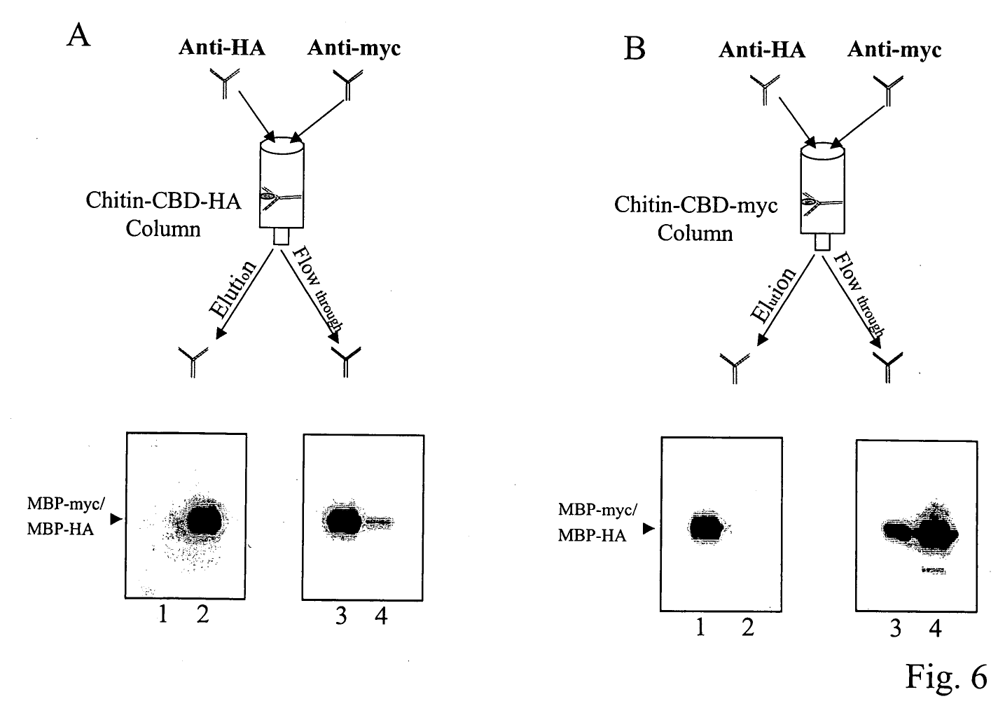

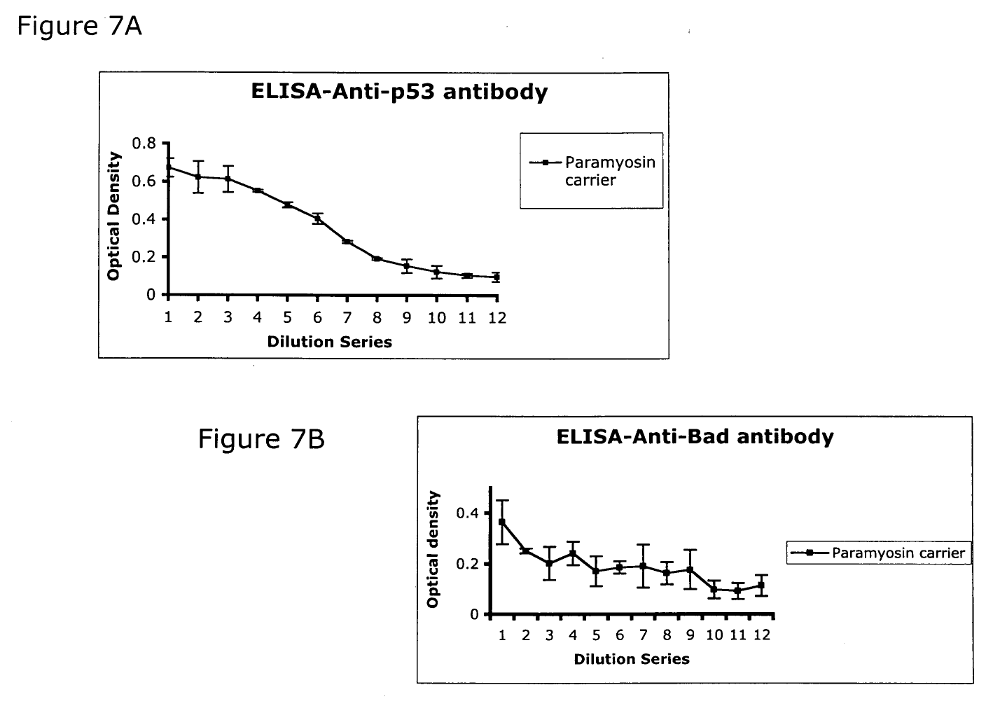

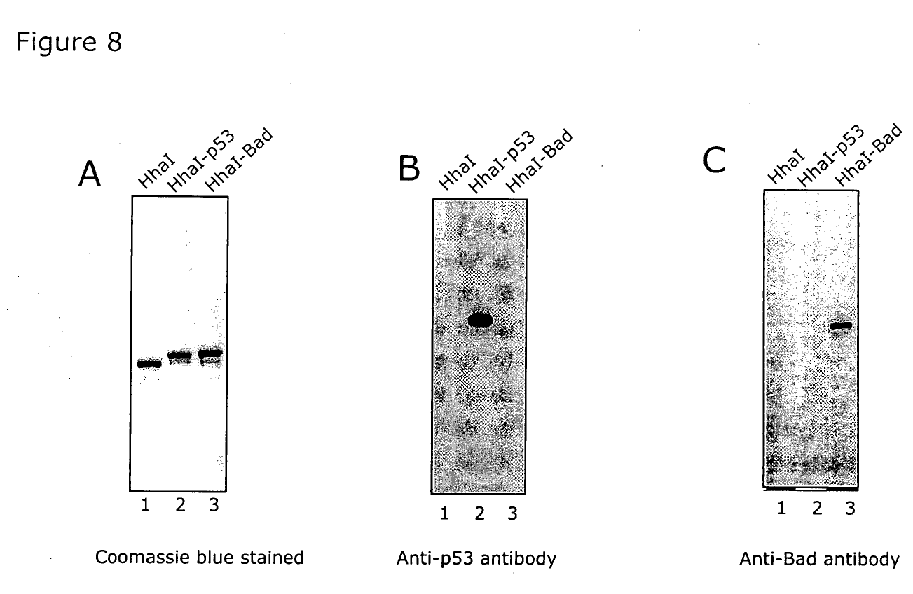

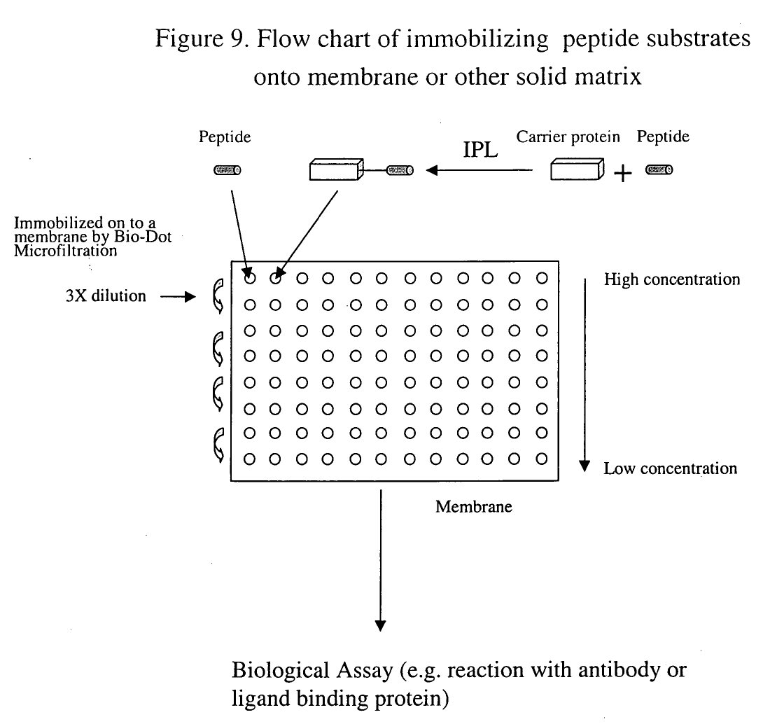

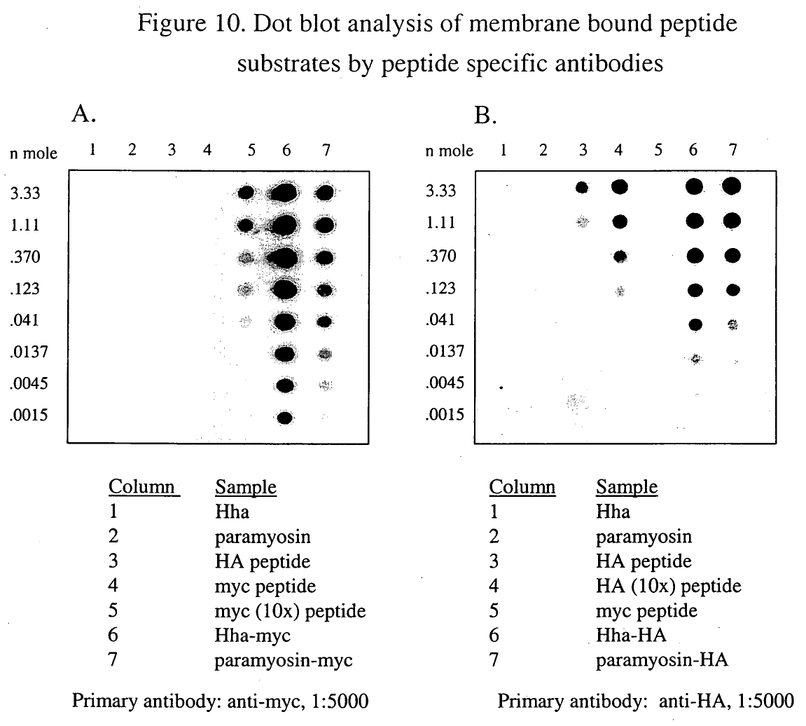

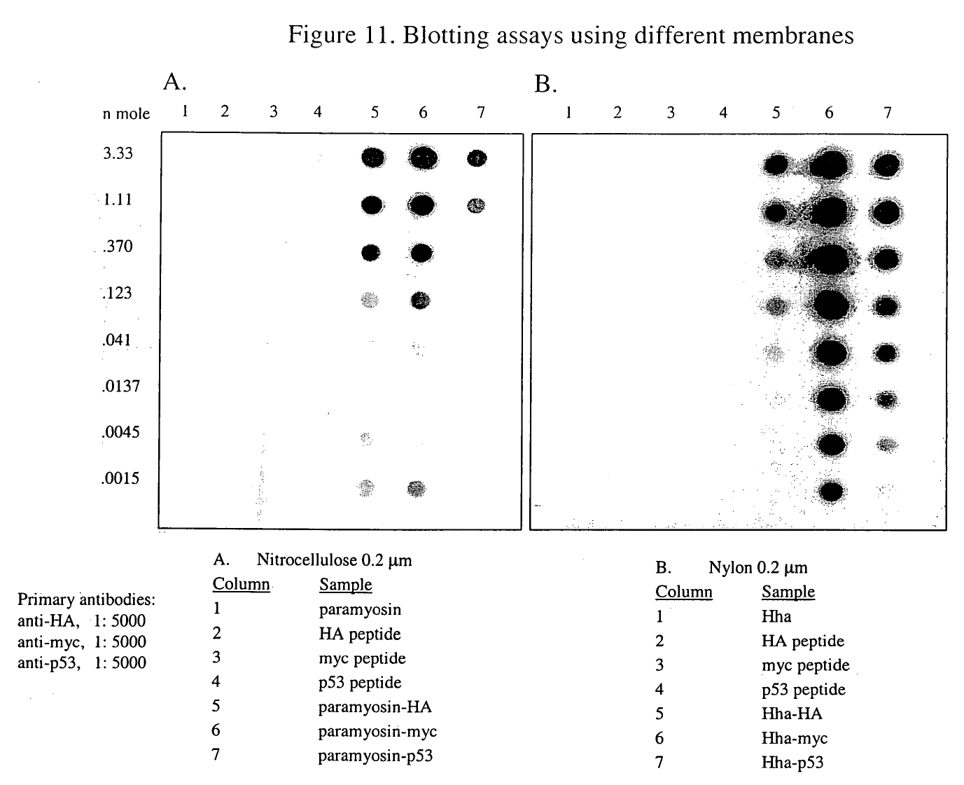

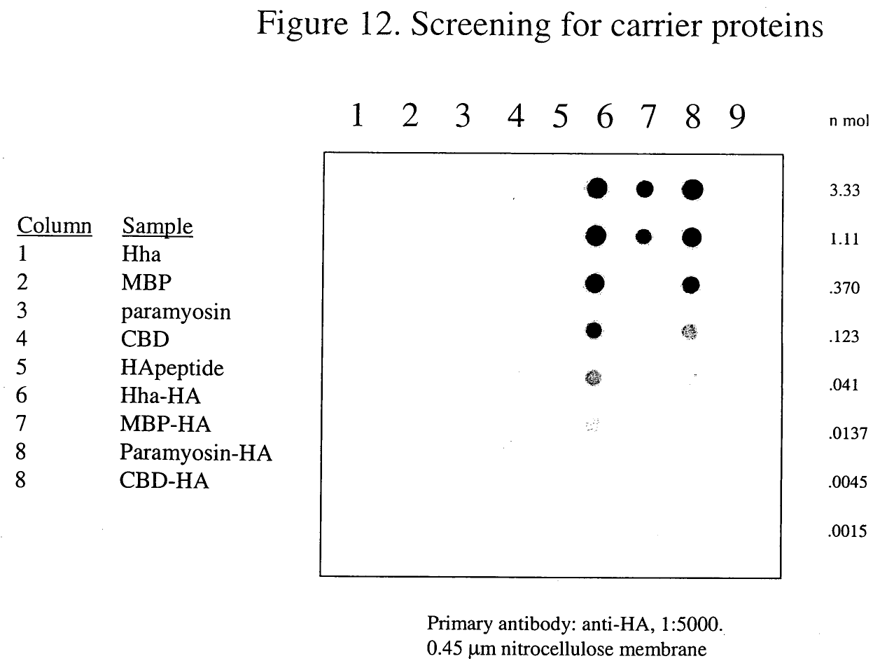

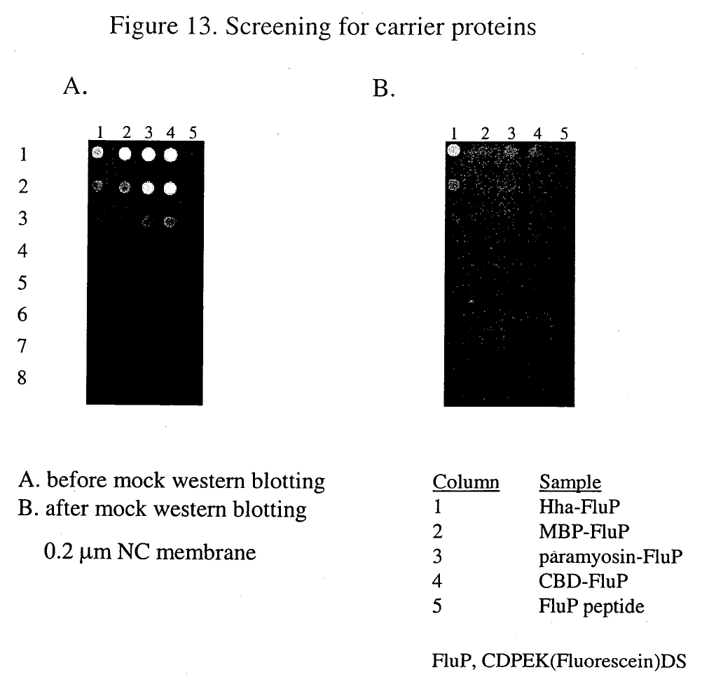

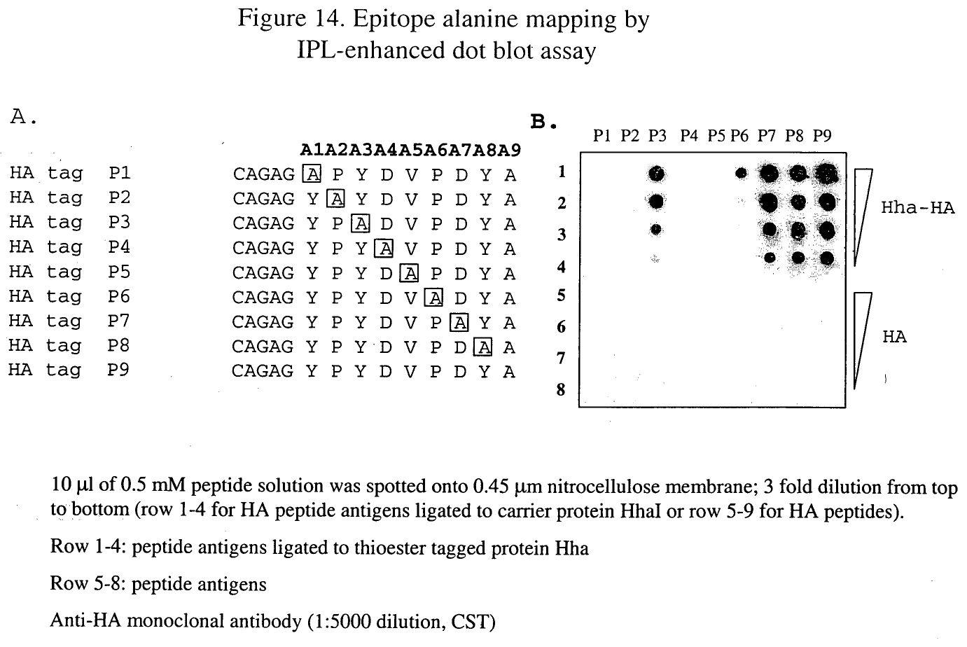

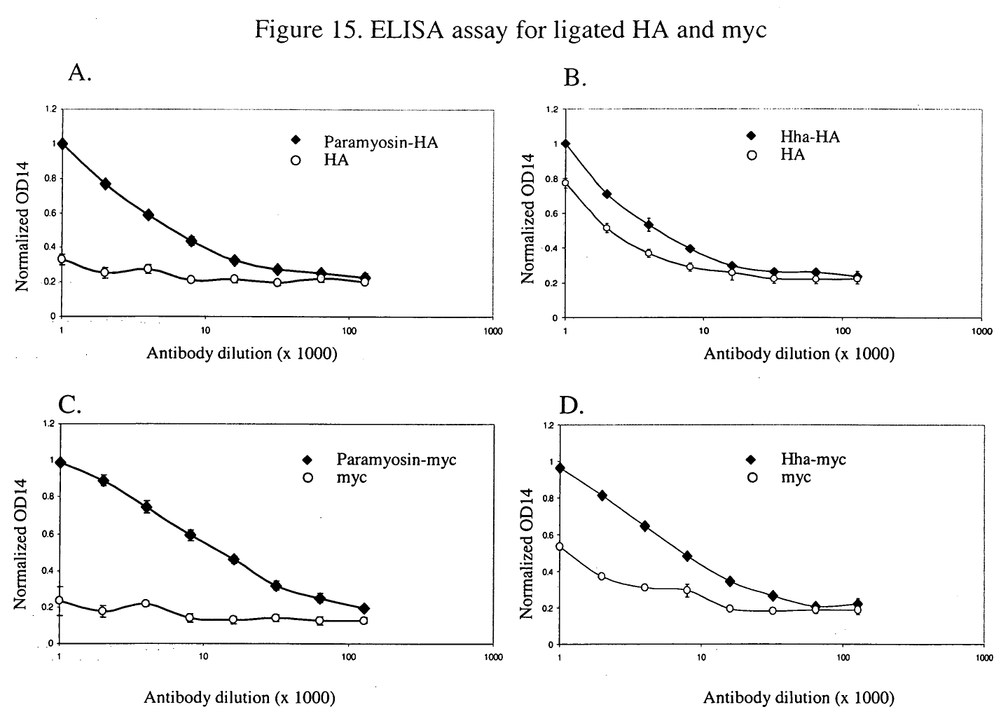

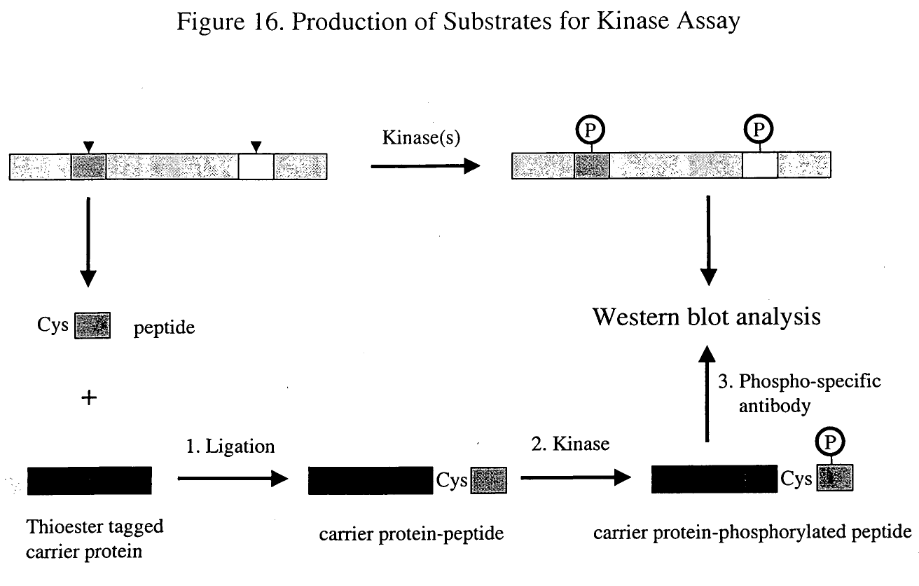

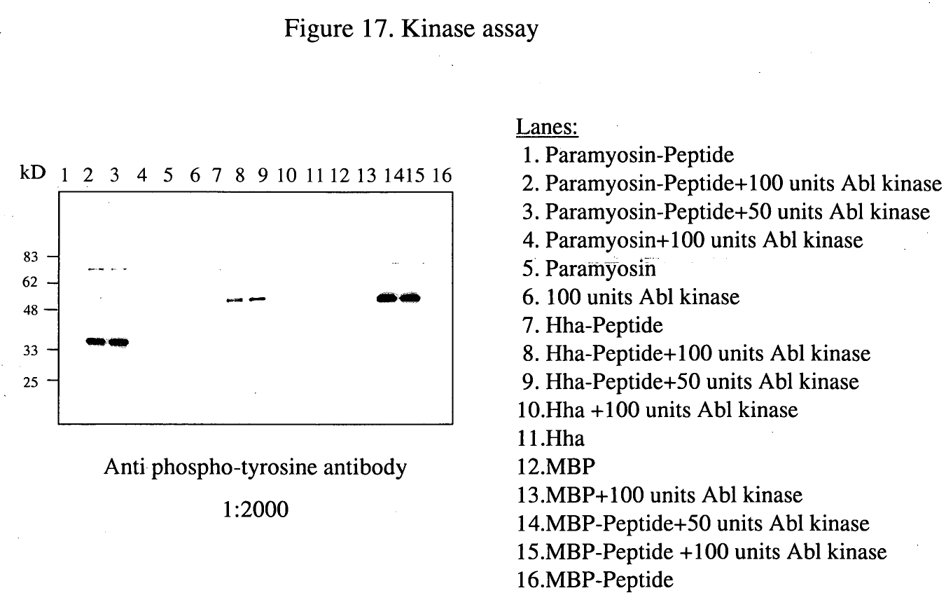

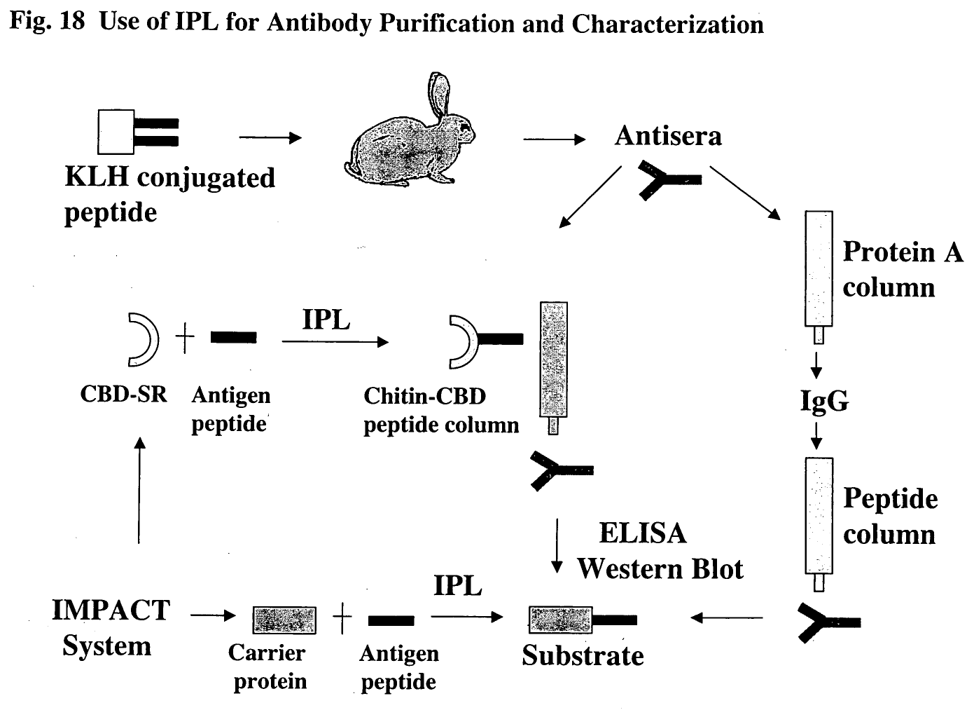

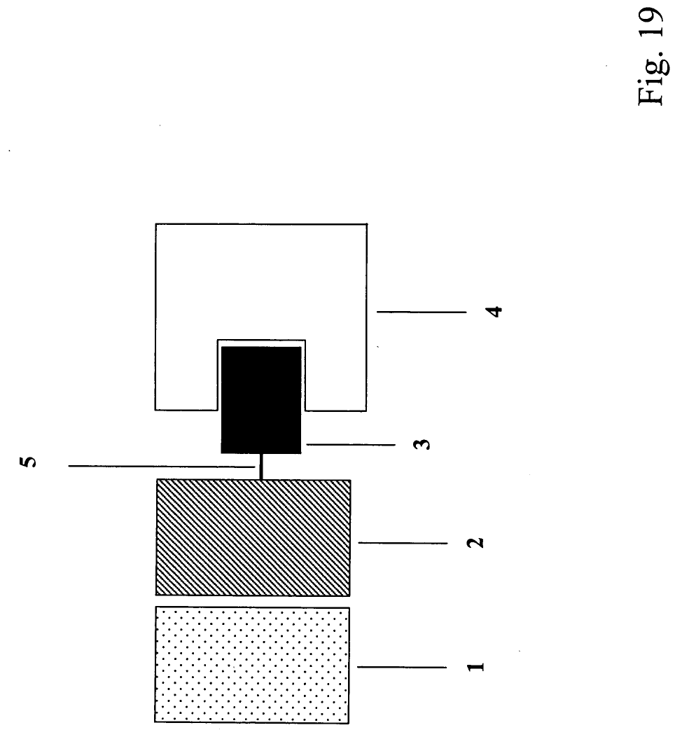

The CBD-intein-CBD* was formed and the intein-mutant CBD was cleaved away to leave a thioester at the C-terminal of the CBD as follows: The chitin column was prepared by equilibrating 2 ml CBD-intein-CBD* bound chitin resin with 5 column volumes of column buffer (500 mM NaCl, 20 mM Tris-HCl, pH 8.5). 40 mM MESNA was added to 30-100 mM final concentration and a thioester intermediate was formed with more than 95% efficiency yielding intein-CBD* and the wild-type CBD fragments. CBD and CBD* were distinguishable on the basis that CBD* binds reversibly to chitin while CBD binds irreversibly. After washing away the intein-CBD* fragment from the column, CBD bound to the resin was efficiently ligated to the HA antigen peptide having an N-terminal cysteine to produce CBD-HA (B-HA) (FIG. 2).

Peptide Sequence

Peptide antigens were synthesized with an additional cysteine residue at their N-terminus. The peptides were synthesized using an ABI model 433A peptide synthesizer, using FastMoc chemistry (Fields, et al. Peptide Research 4: 95-101 (1991)). FMoc/NMP chemistry using HBTU amino acid activation was employed for all cycles (Dourtoglou, et al. Synthesis 572-574 (1984); Knorr, et al. Tetrahedron Letters 30:1927-1930 (1989). Antisera were raised in rabbits against peptides corresponding to residues Ser⁹ to Lys²² (CSVEPPLSQETFSDK) (SEQ ID NO:2) of human p53, Tyr⁹⁸ to Ala¹⁰⁶ (CYPYDVPDYA) of hemagglutinin (HA) protein and Glu⁴¹⁰ to Lys⁴¹⁹ (CEQKLISEEDL) (SEQ ID NO:3) of human c-myc. Antibodies to Thr¹⁰⁶ to Leu¹²⁷ (CTRSRHSSYPNEYEEDEEMEEEL) (SEQ ID NO:4) of mouse Bad (anti-Bad) were raised in mice (Covance Research Products Inc., Denver, Pa.).

Peptide Ligation

Ligation of the CBD, possessing a C-terminal thioester to an antigen peptide possessing a N-terminal cysteine was achieved by adding peptide antigen (HA, -p53, myc or Bad) to 0.25-1.0 mM final concentration to form chitin: CBD-HA, chitin: CBD-p53; chitin: CBD-myc; or chitin: CBD-Bad. The reaction was carried out overnight at 4° C. Next day, the column was rinsed with 10 column volumes of column buffer, then loaded with 1 column volume of L-Cysteine solution (50 mM in column buffer, Sigma, St. Louis, Mo.), and incubated at room temperature for 30 minutes. The column was washed with 10 column volumes of 1 M NaCl followed by 10 column volumes of Phosphate Buffered Saline (PBS; 0.9% NaCl, 10 mM potassium phosphate, pH 7.2).

Stability of Chitin:CBD-Antigen Under Various pH Conditions

The CBD-HA (B-HA) was tested for binding to chitin in 0.1 M glycine buffer with a pH range 1.8-3.5 to determine binding stability under conditions for eluting antibody. FIG. 2 shows that B-HA remained bound on the chitin beads after washing with 0.1 M glycine buffer in the pH range of 1.8 to 3.5. No B-HA polypeptide was detected in the eluant. These results demonstrate that the binding of B-HA was not disrupted by 0.1 M glycine over a pH range that is typically used to elute antibody, suggesting that the chitin bound CBD-peptide fusion is a feasible solid matrix for generating an affinity column to purify antibodies.

Antibody Purification Using the Chitin:Chitin-Binding Domain Antigen Affinity Column

1 ml of antiserum in 3 ml of PBS was loaded onto a column packed with 1 ml of chitin resin and the flow-through was re-loaded onto the column two more times. The column was washed with 10 column volumes of 1 M NaCl followed by 10 column volumes of PBS. The antibody was eluted by passing 0.1 M Glycine (pH 2.5-3.0) through the column and the first 5 fractions of 1 ml each collected, and subsequently 100 μL of 1 M Tris HCl, pH 8.5 was added to each ml of eluted antibody.

Western Blot Analysis of Purified Antibody

To determine whether the CBD-peptide affinity resin was suitable for antibody purification, CBD-HA fusion was generated as described above. One ml of the CBD-HA fusion protein bound to chitin resin was used as an antigen affinity substrate to purify 1 ml of anti-HA rabbit crude antiserum. Simultaneously, as a positive control, a standard conjugated HA peptide affinity agarose resin was also prepared (Hermanson et al. Immobilized Affinity Ligand Techniques, pub. Academic Press, CA (1992)). The first 5 ml of antibodies eluted from each column were examined by Western Blot analysis (Harlow and Lane, Antibodies, Cold Spring Harbor Laboratory, Cold Spring Harbor, N.Y. (1988)) to determine their activity.

Western Blot was performed as follows: 200 ng MBP-peptide fusion (MBP-HA as described in Examples II and III) was run on a 12% SDS-PAGE gel then transferred onto nitrocellulose membrane and the membrane was blocked with 5% nonfat dry powder milk in TBSTT (Tris-buffered saline with 0.2% Tween 20 and 0.05% Triton 100, pH 7.5) for 1 hour at room temperature. Primary antibody was added to 2% milk powder in TBSTT at a 1000-fold dilution, or as indicated in the figures, and the membrane was incubated in this solution at 4° C. overnight. The Blot was washed with TBSTT three times for 15 minutes each at room temperature. The secondary antibody, HRP conjugated anti-rabbit or anti-mouse (Cell Signaling Technology, Inc., Beverly, Mass.), was diluted 1:5000 in 2% milk powder in TBSTT and the Blot was incubated in this solution for 1 hour at room temperature, followed by three washes each of 15 minutes in TBSTT. The Blot was developed using the Phototope-HRP Western Blot Detection System (Cell Signaling Technology, Inc., Beverly, Mass.).

The results show that the antibodies purified from both columns have similar anti-HA activity (FIG. 3). Both resins had essentially the same binding efficiency, since the flow through collected from both columns had only marginal anti-HA activity (FIG. 3, lanes 2 and 4). Similar results were obtained in additional experiments using the chitin bound CBD-peptide antigen fusions to purify antibodies from rabbit or mouse sera raised against myc, p53 or Bad peptide antigen.

Analysis of Antibodies Using ELISA

Purified antibodies can be analyzed using ELISA (Harlow and Lane, Antibodies, Cold Spring Harbor Laboratory, Cold Spring Harbor, N.Y. (1988)).

Throughout the present examples, although the primary and secondary antibodies were varied as specified, the ELISA assay was performed as follows: the polystyrene microtiter plate was coated with 100 μl per well of a 1 μM peptide solution, followed by incubation at 37° C. overnight. The plate was then washed three times with PBS containing 0.05% Tween 20 (PBST) and blocked with 200 μl of 4% milk powder in PBS. The plate was incubated at room temperature for 1 hour, washed thrice with PBST and patted dry. 100 μl of primary antibody was added to each well at the recommended dilution in 2% milk powder in PBS. After adding the antibody, the plate was incubated at 37° C. for 1 hour or room temperature for 2 hours with mild shaking, then washed three times with PBST and patted dry. Secondary antibody at 100 μl per well was used at the recommended dilution in 2% milk powder in PBS, followed by incubation at 37° C. for 1 hour or room temperature for 2 hours with mild shaking. Then, the plates were washed three times with PBST before being developed with 100 μl per well of ABTS (2,2′-Azino-bis[3-ethylbenzothiazoline-6-sulfonic acid], Sigma, St. Louis, Mo.) substrate solution (20 mM ABTS, 0.12% hydrogen proxide, 50 mM citrate buffer, pH 4.0) and read at OD₄₁₄.

ELISA was performed to quantitatively determine and compare the antibody titers after purification from CBD-peptide affinity columns and conjugated-peptide affinity columns. Assays using anti-myc, anti-HA, anti-p53 and anti-Bad antibodies purified by both types of columns indicated that there was no significant difference in their antigen specific activities (FIG. 4). This data is consistent with the results from the Western Blot analysis.

Comparison of Antibodies Purified by Affinity Binding Domain-Ligand Columns with Antibodies Purified from Commercially Available Affinity Purification Columns

In order to assess whether the carrier-ligand (CBD-antigen) affinity column was comparable to commercially available affinity purification products, antibodies from anti-p53 serum was purified on (i) a chitin-CBD-p53 column and (ii) two commercially available affinity column kits: crosslinked agarose support with immobilized iodoacetyl, which reacts with sulfhydryl groups or an N-terminal cysteine (Sulfolink: Pierce Biotechnology, Inc., Rockford, Ill.); beaded agarose support which is activated so that aldehyde functional groups react with primary amines to immobilize antigen peptide (Aminolink: Pierce Biotechnology, Inc., Rockford, Ill.). In this example, the matrix is chitin, the carrier is CBD and the ligand is an antigen where the antigen is p53.

(i) Purification of antibodies on a chitin-CBD-p53 column was achieved by passing antisera over the column and washing the column before eluting the antibodies in a buffer of 0.1 M glycine, pH2.5

(ii) Purification of antibodies from agarose columns was achieved as follows: Agarose beads cross-linked with iodoacetyl groups (Amersham Biosciences, Piscataway, N.J.) were prepared as described previously (Hermanson, et al., Immobilized Affinity Ligand Techniques, pub. Acad. Press, Inc., San Diego, Calif. pp. 75, 99, 118, 143 (1992)). The 2 ml activated agarose column was washed twice with 10 ml binding buffer (0.1 M Tris HCl, pH 8.5, 0.01 M EDTA). A 5 mM peptide solution prepared in binding buffer was incubated with the 2 ml activated agarose column with rotation for 15 min and then stood for a further 30 minutes at room temperature. The column was then washed twice with 10 ml of the binding buffer. 5 ml L-cysteine solution (130 mM) was added to the column and rotated for 15 minutes to allow mixing, then incubated for another 30 minutes at room temperature. Finally 20 column volumes of 1 M NaCl were passed through the column, followed by washing with 10 column volumes of PBS.

Western Blot analysis was used to test the antibody purification efficiency of these three resins. FIG. 5 shows that except for the Aminolink kit, shown in panel C that appears to have marginally better binding efficiency, there is no significant difference in the activity profile of antibodies purified from all three columns. All three columns generated peptide specific antibody with similar titers, as assayed by Western Blot analysis.

Competition Assay to Determine Specificity of Antibody to Antigen

One of the parameters in developing this novel antibody purification strategy is to determine whether the chitin:CBD-antigen affinity resin confers high specificity for purifying an antibody specific for a particular antigen. Crude rabbit anti-HA and anti-myc sera were mixed in a 1:1 ratio and passed through an HA- or an myc-CBD peptide affinity resin (FIG. 6, panels A and B). Flow-through fractions and eluted antibodies were collected and examined by Western Blot analysis. Anti-HA activity was detected only in the elution fraction from the HA affinity column while anti-myc activity was detected only in the flow-through fraction, indicating that the resin specifically retained anti-HA, not anti-myc antibody. Similarly, only anti-myc activity was detected in the elution from the myc peptide affinity column while anti-HA activity was detected only in the flow-through fraction. These results demonstrate that each peptide column specifically retained only those antibodies raised against the peptide antigen present on the resin.

### EXAMPLE II

### Carrier-Antigen Fusions as Immunogens

An immunogen was generated using paramyosin, MBP or M.HhaI as the carrier proteins. The antigen had an N-terminal cysteine while the carrier protein had a C-terminal thioester.

Carrier Preparation

A carrier for ligation to an antigen for forming an immunogen was generated by ligating the peptide antigen having an N-terminal cysteine to a carrier protein possessing a thioester at its C-terminus. The antibody raised in the presence of the immunogen was then analyzed by Western Blot against the antigen conjugated to a second carrier.

The choice of carrier protein for fusion with an antigen to form a reagent for use in Western Blot analysis depends on which carrier protein was used with the antigen to create an immunogen as described. If, for example, paramyosin was used as a carrier protein in the generation of an immunogen for raising antibodies, a carrier protein other than paramyosin was used to create the carrier-antigen reagent for Western Blot analysis of ligand-binding molecules to avoid cross-reactivity and false positives. For example, MBP (U.S. Pat. No. 5,643,758) or M.HhaI (New England Biolabs, Beverly, Mass.) may be used as carrier proteins for ligation of the antigen for Western Blot analysis.

Carrier proteins were obtained by cis- or trans-cleavage of the carrier-intein fusion proteins using a thiol reagent.

Trans-Cleavage to Form a Carrier Protein

MBP was selected as a carrier and expressed as a fusion protein. The fusion protein consisted of MBP, the N-terminal 123 residues of the Ssp DnaE intein and CBD at the C terminus (Evans et al. JBC 275:9091-9094 (2000)). A pKEB1 mutant was constructed to express a fusion protein identified as EB9A. EB9A consisted of the 36 C-terminal amino acids of the Ssp DnaE intein with the terminal intein residue Asp¹⁵⁹ converted into Ala to block the C-terminal cleavage, followed by 3 native extein residues and the CBD at the C terminus. MEB and EB9A fusion proteins were separately expressed and purified on chitin columns following the standard procedure (Evans, et al., Protein Sci. 7(11):2256-2264 (1998); Evans, et al., J. Biol. Chem, 274(7):3923-3926 (1999)). To obtain MBP by trans-cleavage, the chitin beads bound MEB and EB9A fusion proteins were mixed in approximately a 1:1 ratio. MESNA was added to the mixture to a final concentration of 40 mM and the trans-cleavage reaction was carried out at 4° C. overnight. The free MBP bearing a C-terminal thioester was eluted using column buffer (20 mM Tris HCl pH 8.5, 0.5 M NaCl) and ligated to HA peptide (MBP-HA), myc (MBP-myc) or p53 (MBP-p53). The ligation reaction was carried out by adding the peptide (HA, myc or p53) to the final concentration of 0.5 mM at 4° C. to MBP (0.3 μM).

Cis Cleavage to Form a Carrier Protein

Carrier proteins were prepared using the IMPACT™ (New England Biolabs, Inc., Beverly, Mass.) system. A carrier protein was ligated to the antigen similar to the approach described above. For example, MBP or M.HhaI (New England Biolabs, Inc., Beverly, Mass.) were selected as carriers for ligation to peptide antigens, HA (MBP-HA, Hha-HA), myc (MBP-myc, Hha-myc), p53 (MBP-p53, Hha-p53) or Bad (MBP-Bad, Hha-Bad) (Evans, et al., J. Biol. Chem., 274(7):3923-3926 (1999)).

The gene encoding the M.HhaI (37 kD) was transferred from the pCYB (M.HhaI) vector (Chong, et al., Gene. 192:271-281 (1997)) using NdeI and XhoI into pMRB vector (Evans, et al., J. Biol. Chem., 274(7):3923-3926 (1999)) resulting in the fusion of the M.HhaI to the methanobacterium thermoautotrophicum intein (Mth RIR1) and the chitin-binding domain (HRB). To generate the PXB fusion protein, the gene for paramyosin (28 kD D Sal fragment; Limberger, et al. Mol. Biochem. Parasitol. 38:271-280 (1990)) was cloned upstream of the Mxe intein and CBD in a pTXB vector (Evans et al., Biopolymers 51:333-342 (1999).

HRB and PXB fusion proteins were separately expressed and purified on chitin columns following the standard procedure (Evans, et al., Protein Sci. 7(11):2256-2264 (1998); Evans, et al., J. Biol. Chem., 274(7):3923-3926 (1999)). 50 mM MESNA was used to induce cleavage at 4° C. for 12-16 hours and the proteins M.HhaI and paramyosin, possessing C-terminal thioesters, were eluted from the columns in column buffer (20 mM Tris HCl pH 8.5, 0.5 M NaCl). The proteins were then dialysed into a lower pH storage buffer (5 mM Bis-tris, pH 6, 0.5 M NaCl) and stored at −80° C. The paramyosin and M.HhaI were then ligated to the p53 and Bad peptides. The ligation reaction was carried out by adding the peptide (Bad or p53) to the final concentration of 0.5 mM at 4° C. to M.HhaI or paramyosin (at 1 mg/ml) in the presence of a final concentration of 0.1 M Tris, pH 8.5.

The carrier protein of paramyosin linked to the peptides p53 (paramyosin-p53) and Bad (paramyosin-Bad) were then used as immunogens to immunize rabbits (paramyosin-p53) or mice (paramyosin-Bad) (Covance Research Products Inc., Denver, Pa.; Invitrogen, Carlsbad, Calif.).

The polyclonal antibodies for each immunogen, either from two rabbits or from five mice, were pooled and analyzed by ELISA as described above.

The ELISA results showed that p53 or Bad antibodies were effectively produced in inoculated animals when using paramyosin as a carrier protein (FIG. 7), confirming the utility of ligating an antigen to a carrier protein to form an immunogen.

### EXAMPLE III

### Production of a Carrier-Antigen Fusion as a Positive Control for Western Blot Analysis

The small antigens (peptides) are usually not suitable for Western Blot analysis due to their small size. This limitation can be overcome by ligation of the antigen to a carrier protein. The carrier should have a molecular weight sufficient to increase the size of the ligand. For example, the size should preferably be greater than 5,000 Da, more preferably, greater than 10,000 Da for Western Blot analysis.

Carrier Preparation

A carrier for ligation to an antigen for characterization of an antibody by Western Blot analysis or other methods was generated by ligating the antigen with a carrier protein possessing a thioester at its C-terminus as described in Example II.

In this example, M.HhaI was used as a carrier protein to ligate to the antigen peptides p53 and Bad (FIG. 8; for peptide sequences, see Example 1.) The production of M.HhaI carrier and the ligation reactions are described in Example II. M.HhaI-p53 and M.HhaI-Bad fusions were used as positive controls for Western Blot analysis of anti-p53 and anti-Bad antibodies, respectively.

In FIG. 8, polyclonal rabbit anti-p53 antibody was used at 1:15,000 and the secondary antibody, HRP conjugated anti-rabbit antibody (Cell Signaling Technology, Inc., Beverly, Mass.), was used at 1:2500 while the polyclonal mouse anti-Bad antibody was used at 1:7,500 and the secondary antibody, HRP conjugated anti-mouse antibody (Cell Signaling Technology, Inc., Beverly, Mass.), was used at 1:2500. The Western Blots showed that the antibodies recognized a distinct, specific band of the ligated products (Hha-p53 and Hha-Bad respectively), demonstrating the utility of this technique in the generation of a positive control or substrate for evaluation of antibodies.

### EXAMPLE IV

### Enhanced Attachment of Ligand (Peptide) Substrates to Membranes

In this example, carrier-ligand (in this case, antigen peptide) substrates were prepared using intein-mediated protein ligation and dot blotted onto membranes for testing their reactivity against ligand-binding molecules, in this case antibodies. The peptide ligated to a carrier was demonstrated to exhibit significantly higher affinity to various types of membranes than the peptide alone.

Peptides and Antisera

HA corresponding to residues Tyr98 to Ala106 (CYPYDVPDYA) of HA protein; Human c-myc corresponding to Glu410 to Lys419 (CEQKLISEEDL) (SEQ ID NO:5) and Human p53 corresponding to Ser9 to Lys22 (CSVEPPLSQETFSDK) (SEQ ID NO:6) were synthesized with an additional cysteine residue at their N-terminus (New England Biolabs, Beverly, Mass.).

Peptides were synthesized using an ABI model 433A peptide synthesizer, using FastMoc™ chemistry at a scale of 0.085 mmol. Fmoc/NMP chemistry utilizing HBTU amino acid activation was employed for all cycles. (Fields, et al. Peptide Research 4: 95-101 (1991); Dourtoglou et al., Synthesis 572-574 (1984); and Knorr et al. Tetrahedron Letters 30:1927-1930 (1989)).

Antisera were raised against HA, myc and p53 peptides in rabbits (Covance Research Products Inc., Denver, Pa.). The polyclonal antibodies were purified by peptide affinity column (Sun et al. Journal of Immun. Method 282:45-52 (2003)). Monoclonal anti-HA antibody was purchased from Cell Signaling Inc. (Beverly, Mass.). A fluorescent peptide (FluP, CDPEK (Fluorescein) DS) was synthesized with Fluorescein conjugated to the lysine residue (New England BioLabs, Inc., Beverly, Mass.).

Ligation of Peptide to the Carrier Protein

Purified M.HhaI, MBP, paramyosin and CBD carrier proteins containing a C-terminal thioester were ligated to several synthetic peptide antigens including HA, c-myc, p53 and FluP, all synthesized with an N-terminal cysteine. The ligation reactions were carried out in the presence of MESNA (10 mM final concentration), peptide (0.5 mM final concentration), carrier protein (0.02-0.04 mM final concentration) and 100 mM Tris-HCl, pH 8.5. The reaction was carried out overnight at 4° C. The ligation efficiency was determined to be typically 70-90% by comparing an unligated carrier-peptide sample to the ligated carrier-peptide sample on a 12% or 10-20% SDS-PAGE gel, stained with Coomassie Blue.

Dot Blotting Assay

Blotting assays were performed as follows (FIG. 9): 10 μL of a 0.5 mM peptide solution or 10 μL of an IPL reaction (0.5 mM peptide, 0.02 mM carrier protein, 100 mM Tris pH 8.5) were mixed with 140 μL 1×PBS in the first row (Row A) of a 96-well plate. 100 μL of 1×PBS was added to Rows B to H of the plate. Next, 50 μL of solution was transferred from Row A to Row B; this transfer step was repeated for the remaining rows of the plate and the extra 50 μL of solution was discarded from the last row to complete the serial dilution. This yielded a three-fold difference in antigen concentration between adjacent wells. Subsequently, 100 μL of the diluted peptide solution was transferred from the 96 well plate to the corresponding position of the Dot Blot Apparatus under a vacuum (Bio-Dot Microfiltation Apparatus, Biorad, Hercules, Calif.). Primary antibody was applied at the recommended dilution overnight at 4° C. in 2% nonfat dry milk in Tris-buffered saline with 0.2% Tween 20 and 0.05% Triton 100, pH 7.5 (TBSTT) followed by three fifteen-minute washes in TBSTT. Next, secondary antibody was applied at the recommended dilution in 2% dry milk in TBSTT for one hour at room temperature followed by three additional fifteen-minute washes in TBSTT at room temperature. The Blots were then developed using the Phototope-HRP Western Blot Detection System (Cell Signaling Technology, Inc., Beverly, Mass.). Dot Blotting Assays using the Fluorescein labeled peptide (FluP) were conducted according to the procedure described above, in the absence of primary and secondary antibodies (mock Western Blotting), and were visualized with AlphaImager 3400 Software (Alpha Innotech) under UV light (254-366 nm) (FIG. 13).

Protein blotting was performed to transfer the carrier protein-peptide ligation samples to nitrocellulose membranes (0.45 mm). The membranes were incubated with anti-HA or anti-myc antibody. The amount of sample used for the first row was standardized by using the same amount of peptide in each well (FIG. 10). A sample of tenfold concentrated myc or HA peptide (FIG. 10, panel A, column 5 and panel B, column 4, respectively) was included in both blots, to extend the range of the test. After incubation with anti-HA or anti-myc antibodies, ligated peptides produced significantly stronger signals than the unligated peptides. As shown in FIG. 10, the sensitivities for M.HhaI ligated myc and HA peptides increased by approximately 22,200 and 80 fold, respectively, when compared to unligated peptides. For paramyosin ligated myc and HA, the sensitivities increased by 90 and 27 fold, respectively.

Paramyosin or M.HhaI ligated to HA, myc and p53 antigens were immobilized on nitrocellulose (0.2 μm) or a nylon membrane (0.2 μm) (FIG. 11). The carrier-antigen fusion gave a significantly improved signal compared with antigen alone.

Several thioester-tagged carrier proteins were tested for their ability to improve the sensitivity of a peptide dot Blot assay. These carrier proteins (M.HhaI, MBP, paramyosin and CBD) are produced easily in E. coli and have few or no cross reactive epitopes thus reducing the non-specific background in immunoBlots.

After ligation of carrier with HA antigen, protein Blotting was performed to transfer the carrier protein-peptide samples in various dilutions to nitrocellulose membrane. An immunoassay was carried out by incubation of the membrane with anti-HA antibody (FIG. 12). HA ligated to M.HhaI demonstrated the strongest signal against antibody, and HA ligated to CBD demonstrated the least improvement compared to the signal of peptide alone. These results are consistent with previous data (FIGS. 10 and 11).

To directly demonstrate the efficiency of the carrier proteins for immobilizing a peptide onto a membrane, M.HhaI, MBP, paramyosin and CBD, were ligated to a fluorescent peptide with a N-terminal cysteine (FluP). After protein Blotting using nitrocellulose membrane (0.2 μm), the amount of bound FluP on the membrane was visualized under UV light before and after the membrane was subjected to the wash conditions of immunoassays (FIG. 13). Results indicated that every ligated peptide samples were detected on the membrane but the unligated peptide were not detected, suggesting that the binding of the fluorescent peptides to the membrane was significantly improved after ligation to a carrier protein. Furthermore, after the membrane went through the mock Western Blotting steps, M.HhaI ligated fluorescent peptide had the least decrease in its signal, whereas there is a sharp decrease to the signals for other carrier protein-peptide substrates (FIG. 13). This suggested that M.HhaI, as a carrier protein, has improved binding affinity compared with other carrier proteins (FIG. 12). Similar results were also obtained when Nylon and PVDF membranes (0.2 μm) were used as supporting materials.

### EXAMPLE V

### Epitope Mapping by Dot Blot Assay Using Carrier-Peptide Fusion Proteins

Carrier protein-peptide antigen fusions generated by intein-mediated ligation has been used here to create a membrane based peptide array for use in alanine scanning on an HA epitope. Alanine scanning provides a means to map amino acid residues involved in the interaction of antigen with antibody. A peptide array generated by this method was shown to exhibit enhanced sensitivity for detecting antibody binding to antigens.

A group of nine antigens (peptides), all possessing an N-terminal cysteine were used (FIG. 14). The peptide P9 contained the wild-type sequence containing to residues Tyr98 to Ala106 (YPYDVPDYA) of hemagglutinin (HA) protein that can be recognized by a specific antibody. The other 8 peptides carried a single substitution with alanine residue. Each peptide was then ligated to carriers containing a C-terminal thioester by the experimental procedure described in Example II. Subsequently Western Blotting was performed using monoclonal anti-HA antibody (FIG. 14). As a comparison, an array of unligated HA mutant peptides were also dotted onto the same membrane. Results indicated that Ala substitution at position of A7 and A8 had no significant effect on the antibody binding whereas a noticeable decrease was detected when A3 residue was replaced. However, mutation in peptides P1, P2, P4, P5 and P6 had significant effect, suggesting these mutated positions are crucial for mediating peptide antigen-antibody binding. In comparison, however, unligated HA mutant peptides did not generate any measurable signal. The superior signal from ligated HA compared with unligated HA demonstrates the increased sensitivity resulting from ligation of peptide to a carrier.

### EXAMPLE VI

### Enhanced Sensitivity of Signals in ELISA

ELISA assays rely on the absorption of an antigen to a reaction surface. Antibody reacts with the absorbed antigen and a signal is measured in response to an enzyme reaction which is triggered by the binding of antibody to antigen (Harlow and Lane, 1988 ibid.).

Certain peptides may have poor affinity to the solid surface (polystyrene) used for ELISA due to their physical and chemical properties such as molecular mass, charge, hydrophilicity, etc., resulting in low sensitivity and a false positive signal. In this example, when peptides were ligated to a carrier protein, the sensitivity and accuracy of the ELISA assay using these reagents was found to be enhanced.

Myc and HA peptide were ligated to M.HhaI and paramyosin carrier proteins following the procedure in Example II and used as substrates for coating ELISA plates while unligated myc and HA peptides were used as controls.

The data points in FIG. 15 represent the average, normalized OD value of six readings acquired from three independent experiments. The relative activity was obtained by arbitrarily designating the highest OD₄₁₄ value from each reading as “1” and normalizing the data accordingly. The error bars for each point represent the standard deviation for the data, which incorporates experimental uncertainty as well as the measurement uncertainty of the spectrophotometer. The x-axis is a logarithmic scale, while the y-axis is a linear scale. The ELISA data showed that myc-carrier and HA-carrier conjugates produced a stronger signal than unligated peptides as substrates (FIG. 15).

### EXAMPLE VII

### Production of Carrier-Peptide Fusion for Protein Phosphorylation

It is shown here how a carrier-peptide fusion protein can be utilized for analysis of post-translational modifications (FIG. 16). A peptide with a putative phosphorylation site was tested to determine whether a kinase could indeed phosphorylate this site in a carrier-peptide fusion. Phosphorylation was detected using phospho specific antibodies by Western Blot analysis. (FIG. 16). A peptide (Abl peptide), known to be phosphorylated by Abl protein tyrosine kinase (New England Biolabs, Inc., Beverly, Mass.), was synthesized so as to have an N-terminal cysteine. This peptide was then ligated to carrier proteins containing a C-terminal thioester. The ligated product (carrier-peptide) was phosphorylated using Abl protein tyrosine kinase (Abl kinase) (supplied by New England Biolabs, Inc., Beverly, Mass.) and adenosine tri-phosphate (ATP) as a phosphate source. After the kinase treatment, the carrier-peptide samples were subjected to Western Blot analysis with an anti-phospho-tyrosine antibody. Where the Abl kinase was able to phosphorylate the tyrosine of the ligated product, a signal of the ligated product was detected.

Peptide Sequence and Antibody

The peptide Abl peptide, CGSNEAIYAAPFAKKK (SEQ ID NO:7) was synthesized by addition of CGSN at the N-terminus of EAIYAAPFAKKK (SEQ ID NO:9), the substrate of Abl kinase (Songyang, Z. et al. Nature 373:536-539 (1995), (New England Biolabs, Beverly, Mass.). Phospho-tyrosine monoclonal antibody (P-Tyr-100) was purchased from Cell Signaling Technology, Inc., Beverly, Mass.

Ligation of Peptide to the Carrier Protein

The carrier protein HhaI methylase (M.HhaI; 39 kD), from Haemophilus haemolyticus, was purified from a Mth RIR1 fusion, while MBP (42 kD) and paramyosin (28 kD) were purified from Mxe GyrA intein fusions as described in Example II. Ligation of the peptide to the thioester tagged proteins was performed as described in Example II. The ligated products were then subjected to dialysis against water using the Slide-A-Lyzer Mini dialysis units (3,500 MWCO: Pierce Biotechnology, Inc., Rockford, Ill.).

Kinase Assays

Following dialysis the ligated products (also referred to as carrier-peptide conjugates or substrates) were subjected to a kinase reaction using the Abl Protein Tyrosine Kinase (New England Biolabs, Inc., Beverly, Mass.). The substrate (0.7-7 mM final concentration) was reacted with fifty or a hundred units of Abl kinase in the presence of 100 micromolar ATP and 1× Abl kinase buffer (New England Biolabs, Inc., Beverly, Mass.). Controls of unligated protein and negative controls lacking any protein were also set up. The reactions were allowed to proceed at 30° C. for 30 minutes. 3× Loading Buffer (New England Biolabs, Inc., Beverly, Mass.) was added to the samples before they were subjected to Western Blot analysis.

Western Blot Analysis

Western Blot was performed as described above in Example I. Primary antibody (P-Tyr-100; (Cell Signaling Technology, Inc., Beverly, Mass.)) was added to 2% dry milk in TBSTT at a 1000-fold dilution. The secondary antibody, HRP conjugated anti-mouse (Cell Signaling Technology, Inc., Beverly, Mass.), was diluted 1:2500.

The Western Blot analysis demonstrated that a positive signal was detected with the phospho-tyrosine antibody only when a peptide ligated to a carrier protein was treated with Abl kinase (FIG. 17). The carrier-peptide with no kinase treatment, the carrier alone or the kinase alone did not show any detectable signal in the presence of phospho-tyrosine antibody indicating that the phosphorylation site was specific for the kinase-treated peptide and did not occur on the carrier or kinase. When a peptide did not contain a phosphorylation site, then a positive signal was not detected with the phospho-tyrosine antibody in Western Blot analysis or ELISA.

Using radioactive phosphate, phosphorylation in the presence of a kinase can result in the formation of a radioactive peptide-carrier which maybe easily detected by a phosphorylation assay according to the method previously described (Northwood, I. C. et al. J. Biol. Chem. 266:15266-15276 (1991)).

### EXAMPLE VIII

### Preparation of Bioconjugate Cys-Aminohexyl-CGCTCTAGAACTAGTGGATC (SEQ ID NO: 8)

A non-protein ligand was chemically modified so as to place a cysteine at one end of the DNA for reacting with a reactive thioester on a carrier. IuM 1000A C-resin was used with MMT-aminohexylphosphoramidite from Glen Research, Sterling, Va. Detrilation was accomplished by multiple DMT-OFF cycles until no further color appeared (6-8 cycles). Resin was dried and a slurry was formed in dry DMF for about 15 minutes. The product was allowed to settle in 10 ml test tube. A solution of Fmoc-cys (MMT)-OH was added followed by DIPEA and allowed to react for 1 hour. DMF was decanted off and washed repeatedly with additional DMF followed by one ml aliquots of methylene chloride (CH₂CL₂) to wash the mixture repeated about eight times. TCA in CH₂CL₂ was used to detritylate. The resin was washed with ethanol. The concentrate ammonium hydroxide was added and heated 55° C. overnight. The product was worked up as normal DNA. A DNA-cysteine aminohexyl conjugate was successfully obtained.

1 uM resin, 305 mg protected cysteine, 1 ml HOBT/HBTU, 3 ml DMF for 5 mins. 0.5 ml 2M DIPEA in NMP. 500 fold excess Cysteine

What is claimed is:

1. A method for purifying a ligand-binding molecule from a mixture, comprising:
(a) forming a carrier-ligand conjugate by means of a thioester-nucleophile reaction between a carrier reagent and a ligand, the carrier-ligand being optionally immobilized on a matrix;
(b) contacting the carrier-ligand conjugate with a mixture containing the ligand-binding molecule;
(c) selectively binding the ligand-binding molecule to the ligand; and
(d) eluting the ligand-binding molecule from the immobilized ligand so as to obtain the purified ligand-binding molecule.

2. A method according to claim 1, wherein the ligand-binding molecule is an antibody and the mixture is an antiserum.

3. A method according to claim 1, wherein the carrier is a matrix-binding molecule.

4. A method according to claim 1, wherein the matrix-binding molecule is selected from the group consisting of: a monosaccharide-binding domain, a disaccharide-binding domain, an oligosaccharide-binding domain, a chitin-binding domain, a maltose-binding domain, an arabinose-binding domain, an arabinogalactan-binding domain, a lectin-binding domain, a vitamin binding-domain, a nucleic acid-binding domain, an amino acid-binding domain, a metal-binding domain, a receptor-binding domain, a sulfate-binding domain and a phosphate-binding domain.

5. A method according to claim 1, wherein the matrix-binding molecule is a carbohydrate binding molecule.

6. A method according to claim 5, wherein the carbohydrate binding molecule is chitin-binding domain.

7. A method according to claim 1, wherein the matrix is chitin, the matrix-binding protein is a chitin-binding domain, the ligand is an antigen and the ligand-binding molecule is an antibody.

8. A method according to claim 1, wherein the ligand is selected from an antigen, an antibody, a receptor, a receptor-binding domain, an enzyme and an enzyme substrate.

9. A method for forming an affinity matrix for binding a ligand-binding molecule, comprising:
(a) forming a C-terminal thioester by cleavage of a fusion protein wherein the fusion protein comprises a carrier fused to an intein or a ligand fused to intein, such that cleavage occurs in the presence of a thiol reagent at the intein junction with the carrier or the ligand;
(b) combining in a mixture, either (i) the carrier with the C-terminal thioester and the ligand with an N-terminal cysteine or selenocysteine or (ii) the carrier with an N-terminal cysteine or selenocysteine and the ligand with the C-terminal thioester; and
(c) permitting the carrier to bind the matrix to form with the ligand after ligation, the affinity matrix.

10. A method according to claim 9, wherein the carrier is selected from the group consisting of: a monosaccharide-binding domain, a disaccharide-binding domain, an oligosaccharide-binding domain, a chitin-binding domain, a maltose-binding domain, an arabinose-binding domain, an arabinogalactan-binding domain, a lectin-binding domain, a vitamin binding-domain, a nucleic acid-binding domain, an amino acid-binding domain, a metal-binding domain, a receptor-binding domain, a sulfate-binding domain and a phosphate-binding domain.

11. A method according to claim 9, wherein the carrier is selected from M.HhaI, paramyosin, CBD and MBP.

12. A ligand-binding molecule affinity matrix comprising:
a carrier conjugated to a ligand by means of a thioester-nucleophilic interaction; wherein one of the carrier or ligand has a nucleophilic group and the other has a reactive thioester group, the carrier-ligand conjugate being immobilized on a matrix, and the ligand in the carrier-ligand being optionally capable of reversibly binding a ligand-binding molecule.

13. A ligand-binding affinity matrix according to claim 12, wherein if the carrier-ligand is immobilized non-specifically, the carrier is selected from M.HhaI, Paramyosin, CBD and MBP.

14. A method for screening for the interaction of one or more immobilized ligands with one or more ligand-binding proteins in a preparation comprising:
(a) covalently linking a carrier to a ligand by means of thioester-nucleophilic interaction to form a carrier-ligand conjugate;
(b) permitting the carrier-ligand to be immobilized by a matrix;
(c) reacting a preparation containing one or more ligand-binding proteins to the carrier-ligand; and
(d) detecting the binding of the one or more ligand-binding protein with the one or more immobilized ligands.

15. A method according to claim 14, wherein the carrier protein is M.Hha.

16. A method according to claim 14, wherein the carrier protein is paramyosin.

17. A method according to claim 14, wherein the carrier protein is selected from chitin-binding domain and maltose-binding protein.

18. A method according to claim 14, wherein the preparation contains one or a mixture of proteins.

19. A method according to claim 14, wherein the preparation contains an affinity purified antibody.

20. A method according to claim 14, wherein the matrix is selected from nitrocellulose and nylon.

21. A method according to claim 14, wherein the matrix is an SDS-polyacrylamide gel.

22. A method according to claim 14, wherein the matrix is a synthetic polymer.

23. A method according to claim 22, wherein the synthetic polymer is a polystyrene micro-titer plate.

24. A method according to claim 14, wherein the preparation contains a single ligand-binding molecule or a set of ligand-binding molecules such that only one type of ligand-binding molecule in the mixture binds to the carrier-ligand.

25. A method according to claim 14, wherein the carrier-ligand is one of a set of fusion proteins such that each fusion protein contains a different ligand fused to a carrier, the fusion proteins being located on the matrix in an ordered array for detecting interactions between ligand and ligand-binding molecules.

26. A method according to claim 14, wherein the fusion protein has a combined molecular weight in greater than 5,000 Da.

27. A method according to claim 14, wherein the ligand is a protein having one or more post-translational or chemical modifications such that the ligand-binding domain is specific for the modified protein.

28. A method for enhancing the immunogenic properties of a ligand such as a peptide antigen in a animal, comprising:
(a) forming a carrier-ligand fusion protein by intein-mediated ligation; and
(b) administering an effective dose of the carrier-ligand fusion protein to the animal to obtain an enhanced immune response compared with the ligand in the absence of the carrier.

29. A ligand-carrier protein fusion molecule, comprising: ligand fused to Hha methylase carrier protein.

30. A ligand-carrier protein fusion molecule, comprising a ligand fused to paramyosin.

31. A method for screening for carrier proteins with affinity to one or more matrices; comprising:
(a) linking each of a plurality of carriers to a ligand by means of a thioester-nucleophilic interaction to form a plurality of carrier-ligands;
(b) permitting the carrier-ligand to bind to a matrix and to a labeled ligand-binding molecule that produces a signal when bound to a matrix; and
(c) comparing the signal on the matrix for the plurality of carriers to determine the affinity of the carriers for the matrix.
